(19) United States
(12) Patent Application Publication
(10) Pub. No.: US 2023/0241259 A1
(43) Pub. Date: Aug. 3, 2023

(54) IMAGING COMPOUNDS SELECTIVE FOR NaV1.7

(71) Applicant: MEMORIAL SLOAN KETTERING CANCER CENTER, New York, NY (US)

(72) Inventors: Junior Gonzales, New York, NY (US); Javier Hernández-Gil, New York, NY (US); Jason S. Lewis, New York, NY (US); Thomas Reiner, New York, NY (US)

(73) Assignee: MEMORIAL SLOAN KETTERING CANCER CENTER, New York, NY (US)

(21) Appl. No.: 18/009,699

(22) PCT Filed: Jun. 8, 2021
(86) PCT No.: PCT/US2021/036338

§ 371 Date: Dec. 9, 2022

Related U.S. Application Data
(60) Provisional application No. 63/037,150, filed on Jun. 10, 2020.

(51) Int. Cl. A61K 51/08

(52) U.S. Cl. CPC A61K 51/088

(57) ABSTRACT

The present technology is directed to compounds useful in the imaging of peripheral neurons.

### CROSS-REFERENCE TO RELATED APPLICATIONS

This application is a U.S. National Phase Application under 35 U.S.C. § 371 of International Application No. PCT/US2021/036338, filed Jun. 8, 2021, which claims the benefit of and priority to U.S. Provisional Application 63/037,150, filed Jun. 10, 2020, the entire contents of each of which are incorporated herein by reference.

### SEQUENCE LISTING

The instant application contains a Sequence Listing which has been submitted electronically in ASCII format and is hereby incorporated by reference in its entirety. Said ASCII copy, created on Jul. 20, 2021, is named 115872-2218_SL.txt and is 2,768 bytes in size.

### FIELD

The present technology is directed to compounds useful in the imaging of peripheral neurons.

### SUMMARY

In an aspect, the resent technology provides a compound of Formula I

(I)
(SEQ ID NO: 1)
χ¹-YCQ-χ²-FLWTCDSERPCCEGLVCRLWC-χ³-IN-NH₂,

or a conservative amino acid substitution variant thereof, a pharmaceutically acceptable salt thereof, and/or a solvate thereof, wherein
[symbol] ¹ is H of the alpha amine of Y, a chelator covalently conjugated to the alpha amine of Y and optionally wherein the chelator is chelating a radionuclide cation (“chelated radionuclide cation”), R¹ covalently conjugated to the alpha amine of Y, or

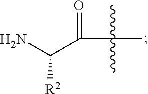

[symbol] ² is

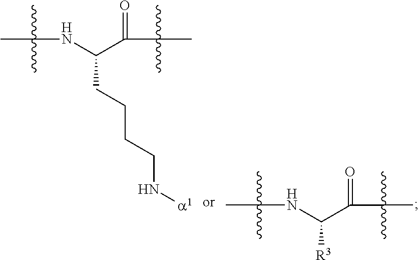

[symbol] is

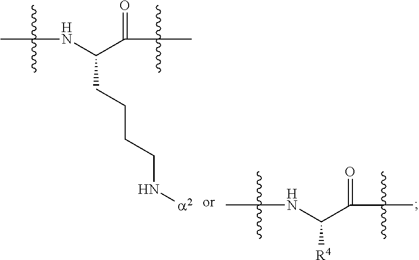

α¹ and α² are each independently H or a chelator optionally chelating a radionuclide cation;
R¹, R², R³, and R⁴ are each independently

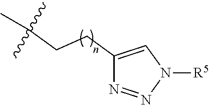

where n is 0, 1, 2, 3, or 4, R⁵ is independently at each occurrence a fluorophore, —(CH₂)ₘ—¹⁸F, an ¹⁸F-substituted aryl, or an ¹⁸F-substituted aralkyl, and m is independently at each occurrence 2, 3, 4, 5, 6, 7, or 8;
provided that [symbol] ¹ is not H of the alpha amine of Y when both α¹ and α² are each independently H.

In an aspect, the present technology also provides compositions that include any aspect or embodiment of a compound of the present technology as disclosed herein and a pharmaceutically acceptable carrier.

In an aspect, the present technology provides pharmaceutical compositions that include an effective amount of a compound of the present technology disclosed herein and a pharmaceutically acceptable carrier.

In an aspect, an imaging method is provided that includes administering a compound of any embodiment herein of the present technology (e.g., such as administering an effective amount) or administering a pharmaceutical composition comprising an effective amount of a compound of any embodiment of the present technology to a subject and, subsequent to the administering, detecting positron emission, detecting gamma rays from positron emission and annihilation (such as by positron emission tomography), and/or detecting Cerenkov radiation due to positron emission (such as by Cerenkov luminescene imaging).

In any aspect or embodiment disclosed herein (collectively referred to herein as “any embodiment herein,” “any embodiment disclosed herein,” or the like) of the compositions, pharmaceutical compositions, and methods including compounds of the present technology, the effective amount may be an imaging-effective amount of the compound for imaging peripheral neurons in a subject.

### BRIEF DESCRIPTION OF THE DRAWINGS

FIGS. 1A-1E illustrate Hsp1a-ChL (a compound of the present technology) accumulation in mouse sciatic nerves. FIG. 1A provides epifluorescence images of animals injected with 100 μL PBS, Hsp1a-ChL (1 nmol, 10 μM of Hsp1a-ChL in 100 μL PBS) or a Hsp1a-ChL/Hsp1a formulation (Hsp1a-ChL, 10 μM, 1 nmol and Hsp1a, 204 μM, 21 nmol in 100 μL PBS), where images were taken 30 min after tail vein injection. FIG. 1B provides a fluorescence intensity quantification of FIG. 1A. FIG. 1C provides epifluorescence images of resected right and left mouse sciatic nerves that were injected with Hsp1a-ChL or [⁶⁴Cu]Cu-ChL-Hsp1a, where high fluorescence intensities (due to dye accumulation) were only observed in sciatic nerves injected with Hsp1a-ChL alone, and no fluorescence was observed after 30 min in mice injected with [[⁶⁴Cu]Cu-ChL-Hsp1a. FIG. 1D provides a fluorescence quantification comparison of animals injected with Hsp1a-ChL and [⁶⁴Cu]Cu-ChL-Hsp1a. FIG. 1E provides bioluminescence quantification of all sciatic nerves (“SN”) injected with [⁶⁴Cu]Cu-Hsp1a versus muscle, spleen, heart, kidney, liver and brain. Statistics were calculated with nonparametric Student's t test. *P<0.05; **P<0.01; ***P<0.001; ****P<0.0001.

FIG. 2 provides the fluorescence of Hsp1a-ChL and Cu-Hsp1a-ChL, showing an increase in fluorescence as an increment in concentration and a decrease in fluorescence signal as different concentration reaction are reacted with CuCl₂.

FIG. 3 illustrates histological validation of Hsp1a-ChL specificity in human vagus nerves after topical application. Human vagus nerve cryosections were stained with the fluorescent Hsp1a-ChL (first and second columns, 3 nmol, 30 μM of Hsp1a-ChL in 100 μL PBS) or with a mixture of Hsp1a and Hsp1a-ChL (third column, Hsp1a-ChL, 30 μM, 3 nmol and Hsp1a, 90 μM, 9 nmol in 100 μL PBS). Human vagus nerves were counterstained with Hoechst 33342 (blue, 20 μM, 1 nmol in 50 μL of PBS). Nerves were sectioned longitudinally, where epineurium and endoneurium can be better observed in composite images

FIG. 4 illustrates a histological validation of Hsp1a-ChL, where fluorescent Hsp1a-ChL uptake was validated in mouse sciatic nerve cryosections from mice injected with Hsp1a-ChL (1 nmol, 10 μM of Hsp1a-ChL in 100 μL PBS) or PBS. Mouse sciatic nerves were sectioned longitudinally.

FIGS. 5A-5D provide the Cerenkov bioluminescence of [⁶⁴Cu]Cu-ChL-Hsp1a in mice, biodistribution, and quantification. FIG. 5A provides Cerenkov Bioluminescence images of mice injected with Hsp1a and [⁶⁴Cu]Cu-ChL-Hsp1a (left) or [⁶⁴Cu]Cu-ChL-Hsp1a alone (right). High radiance is observed after 30 min in mice injected with [⁶⁴Cu]Cu-ChL-Hsp1a alone. FIG. 5B provides a Cerenkov Bioluminescence quantification of FIG. 5A. FIG. 5C provides representative bioluminescence image of sciatic nerves, muscle, spleen, heart, kidney, liver and brain of mice injected with PBS and [⁶⁴Cu]Cu-ChL-Hsp1a. FIG. 5D provides radiance quantification of all sciatic nerves injected with [⁶⁴Cu]Cu-ChL-Hsp1a or Hsp1a and [⁶⁴Cu]Cu-ChL-Hsp1a. Statistics were calculated with nonparametric Student's t test. *P<0.05; **P<0.01; ***P<0.001; ****P<0.0001.

FIGS. 6A-6C provides the results of imaging of fresh, unprocessed, and processed mouse sciatic nerves injected with Hsp1a_Pra0_IR800. FIG. 6A provides epifluorescence images of animals injected with PBS, Hsp1a_Pra0_IR800 (1 nmol, 10 μM of Hsp1a_Pra0_IR800 in 100 μL PBS) or a Hsp1a_Pra0_IR800/Hsp1a_Pra0 formulation (block, Hsp1a_Pra0_IR800, 10 μM, 1 nmol and Hsp1a_Pra0, 30 μM, 3 nmol in 100 μL PBS). Images were taken 30 min after tail vein injection. FIG. 6B provides a fluorescence intensity quantification of unprocessed and processed epifluorescence images of FIG. 6A before and after processing. FIG. 6C provides a fluorescence intensity quantification of FIG. 6A (left) after processing. Parametric unpaired Student's t-test was used to calculate the significance. ****P<0.0001; *P<0.01, ns, non significant. Error bars are standard deviations.

FIGS. 7A-7C provides the results of imaging of fresh, unprocessed, and processed mouse sciatic nerves injected with Hsp1a_Pra0_Janelia669. FIG. 7A provides epifluorescence images of animals injected with PBS, Hsp1a_Pra0_Janelia669 (1 nmol, 10 μM of Hsp1a_Pra0_Janelia669 in 100 μL PBS) or a Hsp1a_Pra0_Janelia669/Hsp1a_Pra0 formulation (block, Hsp1a_Pra0_Janelia669, 10 μM, 1 nmol and Hsp1a_Pra0, 30 μM, 3 nmol in 100 μL PBS). Images were taken 30 min after tail vein injection. FIG. 7B provides a fluorescence intensity quantification of unprocessed and processed epifluorescence images of FIG. 7A before and after processing. FIG. 7C provides a fluorescence intensity quantification of FIG. 7A (left) after processing. Parametric unpaired Student's t-test was used to calculate the significance. ****P<0.0001; **P<0.005, ns, non significant. Error bars are standard deviations.

FIGS. 8A-8C provides the results of imaging of fresh, unprocessed, and processed mouse sciatic nerves injected with Hsp1a_Pra0_BODIPY665. FIG. 8A provides epifluorescence images of animals injected with PBS, Hsp1a_Pra0_BODIPY665 (1 nmol, 10 μM of Hsp1a_Pra0_BODIPY665 in 100 μL PBS) or a Hsp1a_Pra0_BODIPY665/Hsp1a_Pra0 formulation (block, Hsp1a_Pra0_BODIPY665, 10 μM, 1 nmol and Hsp1a_Pra0, 30 μM, 3 nmol in 100 μL PBS). Images were taken 30 min after tail vein injection. FIG. 8B provides a fluorescence intensity quantification of unprocessed and processed epifluorescence images of FIG. 8A before and after processing. FIG. 8C provides a fluorescence intensity quantification of FIG. 8A (left) after processing. Parametric unpaired Student's t-test was used to calculate the significance. ****P<0.0001, ns, non significant. Error bars are standard deviations.

FIGS. 9A-9C provides the results of imaging of fresh, unprocessed, and processed mouse sciatic nerves injected with Hsp1a_Pra0_CY7.5. FIG. 9A provides epifluorescence images of animals injected with PBS, Hsp1a_Pra0_CY7.5 (1 nmol, 10 μM of Hsp1a_Pra0_CY7.5 in 100 μL PBS) or a Hsp1a_Pra0_CY7.5/Hsp1a_Pra0 formulation (block, Hsp1a_Pra0_CY7.5, 10 μM, 1 nmol and Hsp1a_Pra0, 30 μM, 3 nmol in 100 μL PBS). Images were taken 30 min after tail vein injection. FIG. 9B provides a fluorescence intensity quantification of unprocessed and processed epifluorescence images of FIG. 9A before and after processing. FIG. 9C provides a fluorescence intensity quantification of FIG. 9A (left) after processing. Parametric unpaired Student's t-test was used to calculate the significance. *P<0.01, ns, non significant. Error bars are standard deviations.

FIGS. 10A-10C provides the results of imaging of fresh, unprocessed, and processed mouse sciatic nerves injected with Hsp1a_Pra0_DY684. FIG. 10A provides epifluorescence images of animals injected with PBS, Hsp1a_Pra0_DY684 (1 nmol, 10 μM of Hsp1a_Pra0_DY684 in 100 μL PBS) or a Hsp1a_Pra0_DY684/Hsp1a_Pra0 formulation (block, Hsp1a_Pra0_DY684, 10 μM, 1 nmol and Hsp1a_Pra0, 30 μM, 3 nmol in 100 μL PBS). Images were taken 30 min after tail vein injection. FIG. 10B provides a fluorescence intensity quantification of unprocessed and processed epifluorescence images of FIG. 10A before and after processing. FIG. 10C provides a fluorescence intensity quantification of FIG. 10A (left) after processing. Parametric unpaired Student's t-test was used to calculate the significance. ****P<0.0001, ns, non significant. Error bars are standard deviations.

### DETAILED DESCRIPTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, or B and C.”

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Generally, reference to a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P, and ³⁵S are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SF₅), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups may be substituted or unsubstituted. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. The phrase “heteroaryl groups” includes fused ring compounds. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, or a hydroxyl group(s) it can form salts with metals, such as alkali and earth alkali metals (e.g. Na⁺, Li⁺, K⁺, Ca²⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

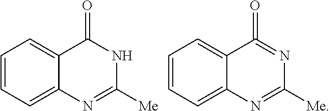

As another example, guanidines may exhibit the following isomeric forms in protic organic solution, also referred to as tautomers of each other:

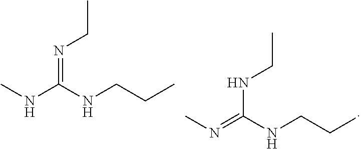

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

As used herein, the terms “subject,” “individual,” or “patient” can be an individual organism, a vertebrate, a mammal, or a human. “Mammal” includes a human, non-human primate, murine (e.g., mouse, rat, guinea pig, hamster), ovine, bovine, ruminant, lagomorph, porcine, caprine, equine, canine, feline, avis, etc. In any embodiment herein, the mammal is feline or canine. In any embodiment herein, the mammal is human.

The term “administering” a compound or composition to a subject means delivering the compound to the subject. “Administering” includes prophylactic administration of the compound or composition (i.e., before the disease and/or one or more symptoms of the disease are detectable) and/or therapeutic administration of the composition (i.e., after the disease and/or one or more symptoms of the disease are detectable). The methods of the present technology include administering one or more compounds or agents. If more than one compound is to be administered, the compounds may be administered together at substantially the same time, and/or administered at different times in any order. Also, the compounds of the present technology may be administered before, concomitantly with, and/or after administration of another type of drug or therapeutic procedure (e.g., surgery).

As used herein, the term “amino acid” is used to refer to any organic molecule that contains at least one amino group and at least one carboxyl group where the at least one amino group is at the a position relative to the carboxyl group, where the amino acid is in the L-configuration. Naturally occurring amino acids include, for example, the twenty most common levorotatory (L,) amino acids normally found in mammalian proteins, i.e., alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val). Naturally-occurring amino acids are those encoded by the genetic code, as well as those amino acids that are later modified, e.g., hydroxyproline, γ-carboxyglutamate, and O-phosphoserine. Another example of a naturally occurring amino acid is L-propargylglycine (Pra), which is produced by certain microorganisms. Amino acids may be referred to herein by either their commonly known three letter symbols or by the one-letter symbols recommended by the IUPAC-IUB Biochemical Nomenclature Commission.

As used herein, the terms “polypeptide,” “polyamino acid,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, “isolated” or “purified” polypeptide, peptide, or protein refers to polypeptide, peptide, or protein that is substantially free of cellular material or other contaminating polypeptides from the cell or tissue source from which the agent is derived, or substantially free from chemical precursors or other chemicals when chemically synthesized. For example, an isolated protein would be free of materials that would interfere with therapeutic uses of the agent. Such interfering materials may include enzymes, hormones and other proteinaceous and nonproteinaceous solutes.

“Treating,” “treat,” “treated,” or “treatment” as used herein covers the treatment of a disease or disorder described herein in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, ameliorating, or slowing progression of one or more symptoms of the disease or disorder. Symptoms may be assessed by methods known in the art or described herein, for example, biopsy, histology, and blood tests to determine relevant enzyme levels, metabolites or circulating antigen or antibody (or other biomarkers), quality of life questionnaires, patient-reported symptom scores, and imaging tests.

“Ameliorate,” “ameliorating,” and the like, as used herein, refer to inhibiting, relieving, eliminating, or slowing progression of one or more symptoms.

As used herein, “prevention,” “prevents,” or “preventing” of a disorder or condition refers to a compound that, in a statistical sample, reduces the occurrence of the disorder, symptom, or condition in the treated sample relative to a control subject, or delays the onset of one or more symptoms of the disorder or condition relative to the control subject.

Throughout this disclosure, various publications, patents and published patent specifications are referenced by an identifying citation. Also within this disclosure are Arabic numerals referring to referenced citations, the full bibliographic details of which are provided subsequent to the Examples section. The disclosures of these publications, patents and published patent specifications are hereby incorporated by reference into the present disclosure to more fully describe the present technology.

### The Present Technology

Injuries to the peripheral nervous system represent a significant concern in surgical practice, and can occur during virtually any type of intervention. While the majority of peripheral nerve injuries occur in the upper limbs and are of traumatic origin, around 25% of patients suffering from neuropathic pain identified surgical intervention as the originating cause. Oncologic surgery in particular carries a considerable risk of peripheral nerve damage because of the distorted physiology around a malignant lesion and the need to achieve complete resection for oncologic control—which, intuitively, increases the likelihood of inadvertent injury.

The present technology provides compounds selective for Na_{V}1.7. Na_{V}1.7 is a sodium channel that is expressed on peripheral neurons and which has received a tremendous amount of attention as a target for analgesics. The compounds of the present technology are useful in the imaging of peripheral neurons, such as by fluorescence emission, positron emission tomography (PET) and/or Cerenkov luminescene imaging.

Cerenkov luminescence imaging (CLI) is an imaging modality for image-guided surgery in general, and especially in regard to surgical margins in particular. CLI is based on the detection of Cerenkov photons emitted by PET imaging agents. Cerenkov photons are emitted by a charged particle (positron or electron) when travelling through a dielectric medium at a velocity greater than the velocity of light in that medium. By detecting the optical photons from PET imaging tracers, CLI combines optical and molecular imaging. CLI with PET agents has been used for in vivo imaging and has rapidly emerged in the field of biomedical imaging. CLI images may be acquired by detecting the Cerenkov light from positron emitting radiotracers using ultra-high-sensitivity optical cameras such as electron-multiplying charge-coupled device (EMCCD) cameras. The CLI image can be analyzed semiquantitatively in photon radiance. Several studies have shown a strong correlation between CLI and PET for different radiopharmaceuticals in vitro, ex vivo, and in vivo.

Thus, in an aspect, the present technology provides a compound of Formula I

(I)
(SEQ ID NO: 1)
χ¹-YCQ-χ²-FLWTCDSERPCCEGLVCRLWC-χ³-IN-NH₂,

or a conservative amino acid substitution variant thereof, a pharmaceutically acceptable salt thereof, and/or a solvate thereof, wherein
[symbol] ¹ is H of the alpha amine of Y, a chelator covalently conjugated to the alpha amine of Y and optionally wherein the chelator is chelating a radionuclide cation (“chelated radionuclide cation”), R¹ covalently conjugated to the alpha amine of Y, or

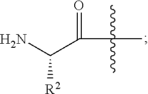

[symbol] ² is

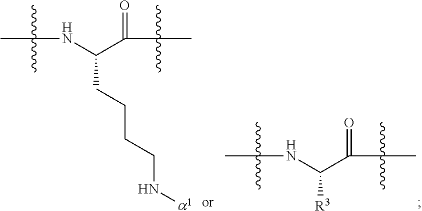

[symbol] ³ is

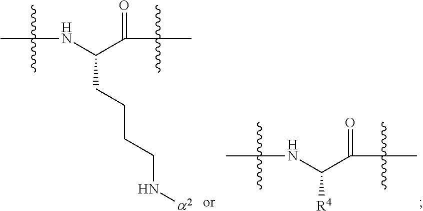

α¹ and α² are each independently H or a chelator optionally chelating a radionuclide cation;
R¹, R², R³, and R⁴ are each independently

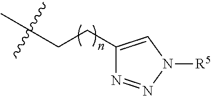

where n is 0, 1, 2, 3, or 4, R⁵ is independently at each occurrence a fluorophore, —(CH₂)ₘ-¹⁸F, an ¹⁸F-substituted aryl, or an ¹⁸F-substituted aralkyl, and m is independently at each occurrence 2, 3, 4, 5, 6, 7, or 8;
provided that [symbol] ¹ is not H of the alpha amine of Y when both α¹ and α² are each independently H.

For clarity's sake, Formula I (SEQ ID NO: 1) is reproduced below with underlining added to the letters that represent the one-letter symbols recommended by the IUPAC-IUB Biochemical Nomenclature Commission for amino acids:

(I)
(SEQ ID NO: 1)
χ¹-YCQ-χ²-FLWTCDSERPCCEGLVCRLWC-χ³-IN-NH₂,

A “conservative amino acid substitution variant” will be well understood by one of ordinary skill in the art. One of ordinary skill in the art understands amino acids may be grouped according to their physicochemical characteristics as follows:
(a) Non-polar amino acids: Ala(A) Ser(S) Thr(T) Pro(P) Gly(G) Cys (C);
(b) Acidic amino acids: Asn(N) Asp(D) Glu(E) Gln(Q);
(c) Basic amino acids: His(H) Arg(R) Lys(K);
(d) Hydrophobic amino acids: Met(M) Leu(L) Ile(I) Val(V); and
(e) Aromatic amino acids: Phe(F) Tyr(Y) Trp(W).

Substitutions of an amino acid in a peptide by another amino acid in the same group are referred to as a conservative substitution (and the resulting peptide a “conservative amino acid substitution variant”) and may preserve the physicochemical characteristics of the original peptide. In contrast, substitutions of an amino acid in a peptide by another amino acid in a different group are generally more likely to alter the characteristics of the original peptide.

In any embodiment disclosed herein, the fluorophore may independently at each occurrence arises from a fluorescent dye such as IR780, IR800, DY-684, DY-700, Janelia669, BODIPY, BODIPY665, sulfo-CY5, CY5.5, CY7, CY7.5, ICG, IR140, or DiR (1,1′-dioctadecyl-3,3,3′,3′-tetramethylindotricarbocyanine). Table A provides an exemplary list of fluorophores that arise from exemplary dyes.

TABLE A

| Fluorophore covalently conjugated to the side chain | Arises From |
|---|---|
| 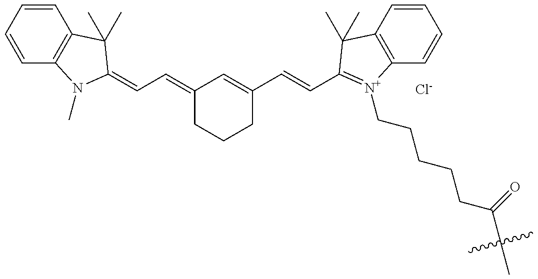 [SMILES: *C(=O)CCCCC[N+]1=C(/C=C/C2=CC(=C/C=C3/N(C)c4ccccc4C3(C)C)/CCC2)C(C)(C)c2ccccc21 ; [Cl-]] | Cy7 (Cyanine 7) |
| 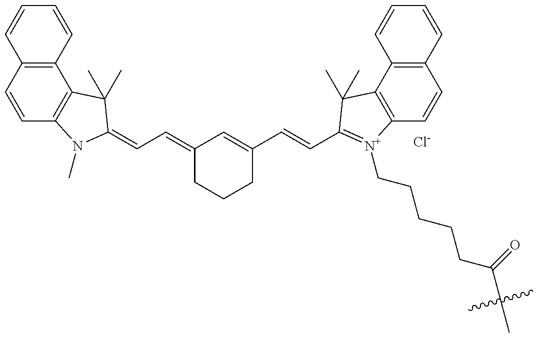 [SMILES: *C(=O)CCCCC[N+]1=C(/C=C/C2=CC(=C/C=C3/N(C)c4ccc5ccccc5c4C3(C)C)/CCC2)C(C)(C)c2c1ccc1ccccc21 ; [Cl-]] | Cy7.5 (Cyanine 7.5) |
| 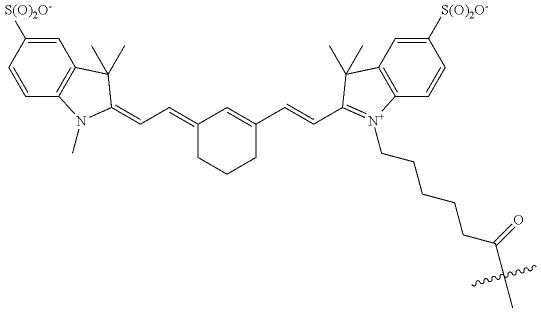 | sulfo-Cy5 |
| 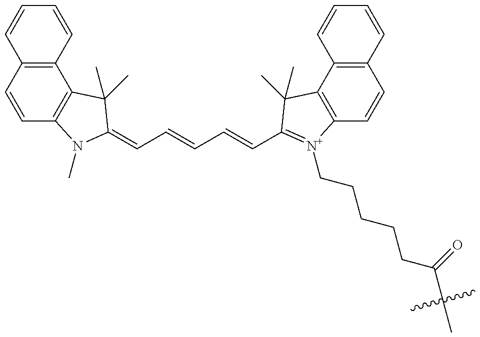 [SMILES: *C(=O)CCCCC[N+]1=C(/C=C/C=C/C=C2/N(C)c3ccc4ccccc4c3C2(C)C)C(C)(C)c2c1ccc1ccccc21] | Cy5.5 |
| 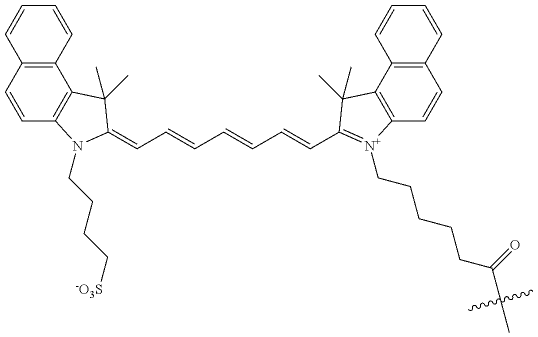 | ICG (Indocyanine green) |
| 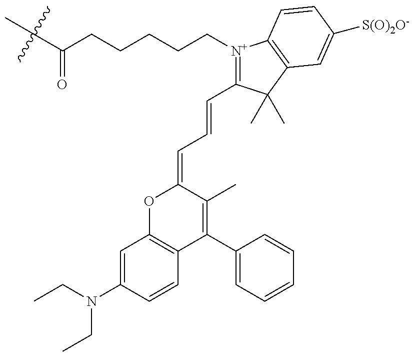 | DY-700 |
| 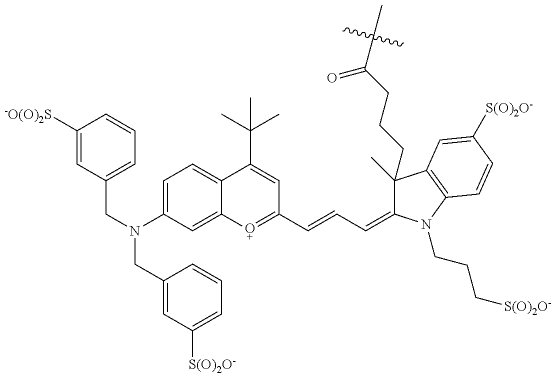 | DY-684 |
| 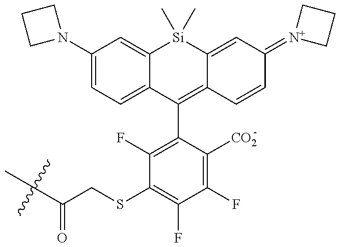 | Janelia669 |
| 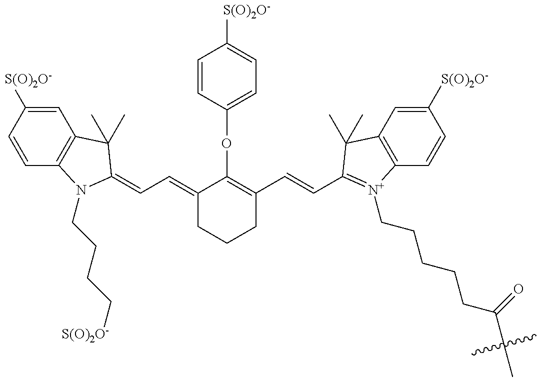 | IR800 |
| 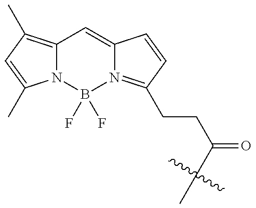 | BODIPY |
| 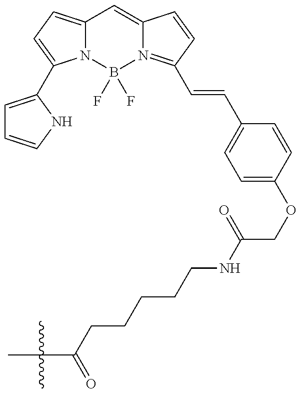 | BODIPY665 |

In any embodiment disclosed herein, it may be the fluorophore is selected from

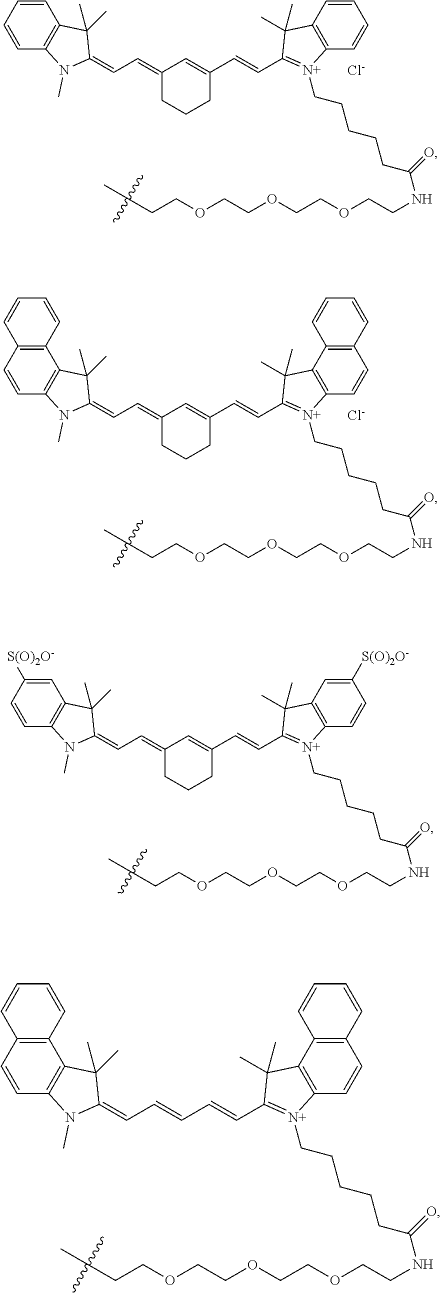

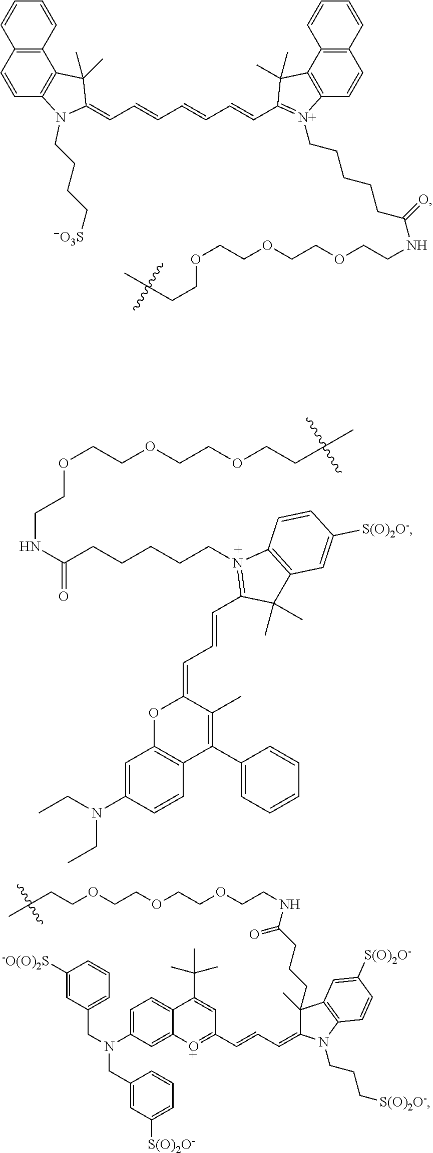

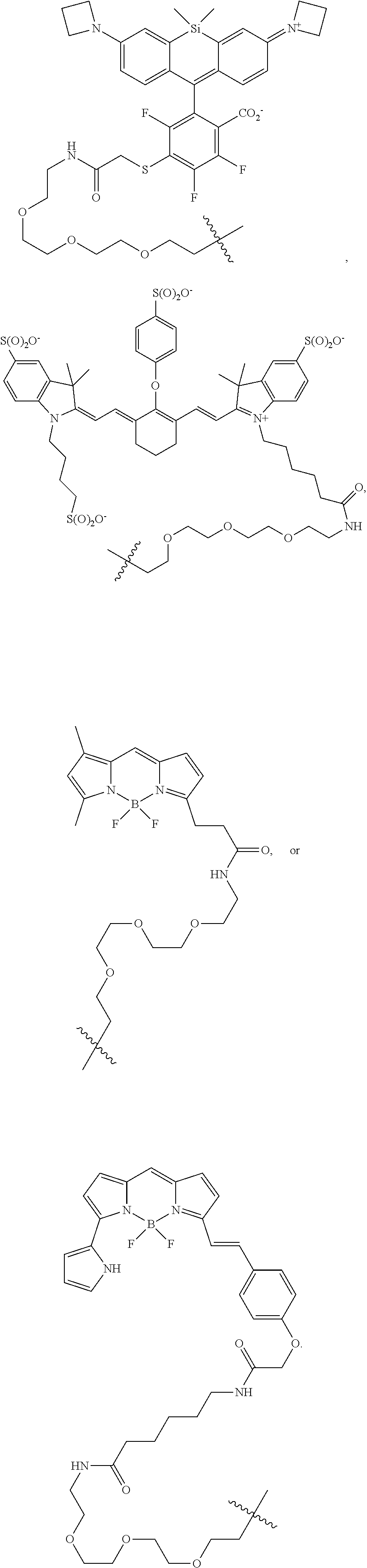

In any embodiment disclosed herein, it may be the chelator is independently at each occurrence

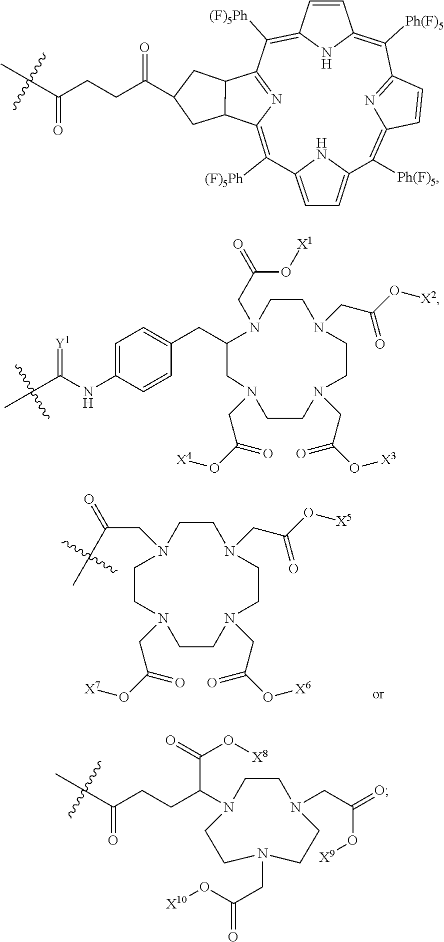

where X¹, X², X³, X⁴, X⁵, X⁶, X⁷, X⁸, X⁹, and X¹⁰ are each independently a lone pair of electrons (i.e., providing an oxygen anion) or H; and Y¹ is S or O. The abbreviation “(F)₅Ph” as used above and herein refers to a pentafluorophenyl group.

In any embodiment disclosed herein, it may be the chelator is chelating a radionuclide cation (“chelated radionuclide cation”) and the chelator and chelated radionuclide cation together is independently at each occurrence

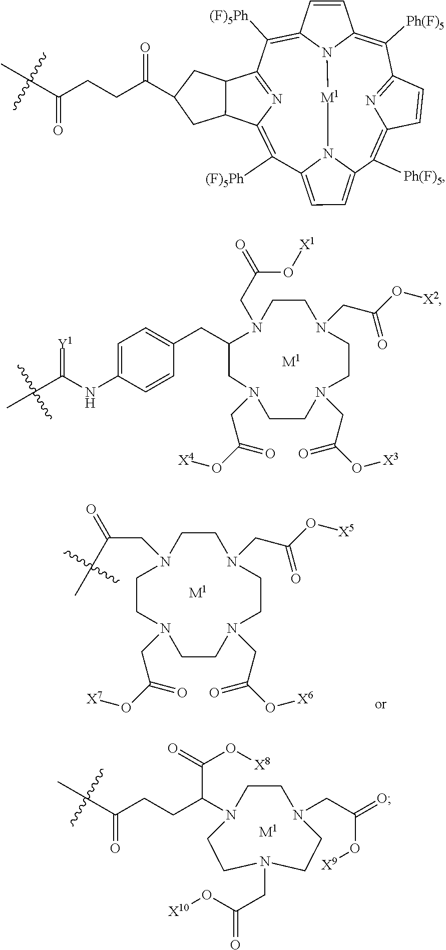

where M¹ is independently at each occurrence a chelated radionuclide cation; X¹, X², X³, X⁴, X⁵, X⁶, X⁷, X⁸, X⁹, and X¹⁰ are each independently a lone pair of electrons (i.e., providing an oxygen anion) or H; and Y¹ is S or O. The abbreviation “(F)₅Ph” as used above and herein refers to a pentafluorophenyl group. The radionuclide cation may be an alpha particle-emitting isotope, a beta particle-emitting isotope, an Auger-emitter, or a combination of any two or more thereof. Examples of alpha particle-emitting isotopes include, but are not limited to, ²¹³Bi, ²¹¹At, ²²⁵Ac, ¹⁵²Dy, ²¹²Bi, ²²³Ra, ²¹⁹Rn, ²¹⁵Po, ²¹¹Bi, ²²¹Fr, ²¹⁷At, and ²⁵⁵Fm. Examples of beta particle-emitting isotopes include, but are not limited to, ⁸⁶Y, ⁹⁰Y, ⁸⁹Sr, ¹⁶⁵Dy, ¹⁸⁶Re, ¹⁸⁸Re, ¹⁷⁷Lu, and ⁶⁷Cu. Examples of Auger-emitters include ¹¹¹In, ⁶⁷Ga, ⁵¹Cr, ⁵⁸Co, ⁹⁹mTc, ^{103m}Rh, ^{195m}Pt, ¹¹⁹Sb, ¹⁶¹Ho, ^{189m}Os, ¹⁹²Ir, ²⁰¹Tl, and ²⁰³Pb. For example, in any embodiment disclosed herein, the radionuclide cation may independently at each occurrence be ⁸⁹Zr, ⁶⁸Ga, ²⁰³Pb, ²¹²Pb, ²²⁷Th, or ⁶⁴Cu. As another example, in any embodiment disclosed herein, the radionuclide cation may independently at each occurrence be ⁸⁹Zr, ⁶⁷Ga, ⁶⁸Ga, ⁸⁶Y, ⁹⁰Y, ⁸⁹Sr, ¹⁶⁵Dy, ¹¹¹In, ¹⁸⁶Re, ¹⁸⁸Re, ¹⁷⁷Lu, ⁶⁷Cu, ⁶²Cu, or ⁶⁴Cu.

¹⁸F containing compounds of the present technology may be provided by reaction of the appropriate ¹⁸F-bearing azide with an intermediate identical to Formula I with the exception that one or more of R¹, R², R³, and R⁴ are each independently

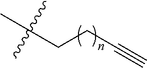

where n is 0, 1, 2, 3, or 4. For example, ¹⁸F containing compounds of the present technology may be generated by a process that includes contacting in the presence of a solvent the intermediate, a copper salt, and an ¹⁸F-bearing azide. For instance, where R⁵ of Formula I is —(CH₂)ₘ-¹⁸F, the ¹⁸F-bearing azide may be N₃—(CH₂)ₘ-¹⁸F. See Marik, J. and Sutcliffe, J. L. “Click for PET: rapid preparation of [¹⁸F]fluoropeptides using Cu^{I} catalyzed 1,3-dipolar cycloaddition” Tetrahedron Letters 2006, 47, 6681-6684. The copper salt may be a copper (I) salt (such as copper (I) iodide), a copper (II) salt (such as copper (II) sulfate), or a mixture thereof. The solvent may be a protic solvent, and aprotic solvent, or a mixture thereof. Protic solvents as used herein include, but are not limited to, alcohols (e.g., methanol (CH₃OH), ethanol (EtOH), isopropanol (iPrOH), trifluoroethanol (TFE), butanol (BuOH), ethylene glycol, propylene glycol), carboxylic acids (e.g., formic acid, acetic acid, propanoic acid, butanoic acid, pentanoic acid, lauric acid, stearic acid, deoxycholic acid, glutamic acid, glucuronic acid), ammonia (NH₃), a primary amino compound (e.g., methyl amine, ethyl amine, propyl amine), a secondary amino compound (e.g., dimethyl amine, diethyl amine, di(n-propyl) amine), water, or a mixture of any two or more thereof. Polar aprotic solvents as used herein include, but are not limited to, ethers (e.g., tetrahydrofuran (THF), 2-methyltetrahydrofuran (2Me-THF), dimethoxyethane (DME), dioxane), esters (e.g., ethyl acetate, isopropyl acetate), ketones (e.g., acetone, methylethyl ketone, methyl isobutyl ketone), carbonates (e.g., ethylene carbonate, propylene carbonate, trimethylene carbonate), amides (e.g., dimethylformamide (DMF), dimethylacetamide (DMA)), nitriles (e.g., acetonitrile (CH₃CN), propionitrile (CH₃CH₂CN), benzonitrile (PhCN)), sulfoxides (e.g., dimethyl sulfoxide, also referred to as “DMSO”), sulfones (e.g., sulfolane), or a mixture of any two or more thereof. For example, the solvent may include DMF and DMSO.

In an aspect, a composition is provided that includes a compound of any aspect or embodiment disclosed herein and a pharmaceutically acceptable carrier or one or more excipients, fillers or agents (collectively referred to hereafter as “pharmaceutically acceptable carrier” unless otherwise indicated and/or specified). In a related aspect, a medicament is provided that includes a compound of any aspect or embodiment disclosed herein. In a related aspect, a pharmaceutical composition is provided that includes (i) an effective amount of a compound of any aspect or embodiment disclosed herein, and (ii) a pharmaceutically acceptable carrier. For ease of reference, the compositions, medicaments, and pharmaceutical compositions of the present technology may collectively be referred to herein as “compositions.” In further related aspects, the present technology provides methods including a compound of any embodiment disclosed herein and/or a composition of any embodiment disclosed herein as well as uses of a compound of any embodiment disclosed herein and/or a composition of any embodiment disclosed herein. Such methods and uses may include an effective amount of a compound of any embodiment disclosed herein.

In a further related aspect, an imaging method is provided that includes administering a compound of any embodiment herein of the present technology (e.g., such as administering an effective amount) or administering a pharmaceutical composition comprising an effective amount of a compound of any embodiment of the present technology to a subject and, subsequent to the administering, detecting positron emission, detecting gamma rays from positron emission and annihilation (such as by positron emission tomography), and/or detecting Cerenkov radiation due to positron emission (such as by Cerenkov luminescene imaging). The detecting step may occur during a surgical procedure on a subject, e.g., to remove a mammalian tissue imaged via the method. The detecting step may include use of a handheld device to perform the detecting step. For example, Cerenkov luminescene images may be acquired by detecting the Cerenkov light using ultra-high-sensitivity optical cameras such as electron-multiplying charge-coupled device (EMCCD) cameras.

In any aspect or embodiment disclosed herein, the effective amount may be determined in relation to a subject. “Effective amount” refers to the amount of a compound or composition required to produce a desired effect. One non-limiting example of an effective amount includes amounts or dosages that yield acceptable toxicity and bioavailability levels for therapeutic (pharmaceutical) use including, but not limited to, the imaging of peripheral neurons. In any aspect or embodiment disclosed herein (collectively referred to herein as “any embodiment herein,” “any embodiment disclosed herein,” or the like) of the compositions, pharmaceutical compositions, and methods including compounds of the present technology, the effective amount may be an imaging-effective amount of the compound for imaging peripheral neurons in a subject. An “imaging-effective amount” refers to the amount of a compound or composition required to produce a desired imaging effect, such as a quantity of a compound of the present technology necessary to be detected by the detection method chosen. For example, an effective amount of a compound of the present technology includes an amount sufficient to enable detection of binding of the compound to peripheral neurons. Another example of an effective amount includes amounts or dosages that are capable of providing a fluorescence emission (above background) in peripheral neurons in a subject, such as, for example, statistically significant emission above background. As used herein, a “subject” or “patient” is a mammal, such as a cat, dog, rodent or primate. Typically the subject is a human. By way of example, the effective amount of any embodiment herein including proteins of the present technology may be from about 0.01 g to about 200 mg of the compound per gram of the composition, and preferably from about 0.1 μg to about 10 mg of the compound per gram of the composition.

The pharmaceutical composition of any embodiment disclosed herein may be packaged in unit dosage form. The unit dosage form is effective in imaging peripheral neurons. Generally, a unit dosage including a compound of the present technology will vary depending on patient considerations. Such considerations include, for example, age, protocol, condition, sex, extent of disease, contraindications, concomitant therapies and the like. An exemplary unit dosage based on these considerations may also be adjusted or modified by a physician skilled in the art. For example, a unit dosage for a patient comprising a compound of the present technology may vary from 1×10⁻⁴ g/kg to 1 g/kg, preferably, 1×10⁻³ g/kg to 1.0 g/kg. Dosage of a compound of the present technology may also vary from 0.01 mg/kg to 100 mg/kg or, preferably, from 0.1 mg/kg to 10 mg/kg. Suitable unit dosage forms, include, but are not limited to parenteral solutions, oral solutions, powders, tablets, pills, gelcaps, capsules, lozenges, suppositories, patches, nasal sprays, injectables, implantable sustained-release formulations, mucoadherent films, topical varnishes, lipid complexes, liquids, etc.

The compositions of the present technology may be prepared by mixing one or more compounds of any embodiment disclosed herein of the present technology with one or more pharmaceutically acceptable carriers in order to provide a pharmaceutical composition useful to prevent and/or treat pain (when proteins of the present technology are included) or useful in imaging peripheral neurons (when compounds of the present technology are included). Such compositions may be in the form of, for example, granules, powders, tablets, capsules, syrup, suppositories, injections, emulsions, elixirs, suspensions or solutions. The instant compositions may be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular injections. The following dosage forms are given by way of example and should not be construed as limiting the present technology.

For oral, buccal, and sublingual administration, powders, suspensions, granules, tablets, pills, capsules, gel caps, and caplets are acceptable as solid dosage forms. These can be prepared, for example, by mixing one or more compounds of the instant present technology with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, enteric coatings, controlled release coatings, binders, thickeners, buffers, sweeteners, flavoring agents, perfuming agents, or a combination of any two or more thereof. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical compositions may be prepared as liquid suspensions or solutions using a sterile liquid, such as, but not limited to, an oil, water, an alcohol, and combinations of these. Pharmaceutically suitable surfactants, stabilizers, antioxidants, suspending agents, emulsifying agents, buffers, pH modifiers, or a combination of any two or more thereof, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as, but not limited to, poly(ethylene glycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Additionally or alternatively, sterile oils may be employed as solvents or suspending agents. Typically, the oil or fatty acid is non-volatile, including natural or synthetic oils, fatty acids, mono-, di- or tri-glycerides.

For injection, the composition may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, buffers, surfactants, bioavailability modifiers, and combinations of any two or more of these.

Compounds of the present technology may be administered to the lungs by inhalation through the nose or mouth. Suitable compositions for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and non-aqueous (e.g., in a fluorocarbon propellant) aerosols may be used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically acceptable carrier and/or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remington's Pharmaceutical Sciences” Mack Pub. Co., New Jersey (1991), and “Handbook of Pharmaceutical Excipients” by Raymond Rowe. Pharmaceutical Press, London, UK (2009), each of which is incorporated herein by reference.

The compositions (e.g., pharmaceutical compositions) of the present technology may be designed to be short-acting, fast-releasing, long-acting, and sustained-releasing as described below. Thus, the compositions may also be formulated for controlled release or for slow release.

The compositions of the present technology may also include, for example, micelles or liposomes, or some other encapsulated form, or may be administered in an extended release form to provide a prolonged storage and/or delivery effect. Therefore, the compositions may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Various assays and model systems, for example those described herein, can be readily employed to determine the therapeutic effectiveness of the treatment according to the present technology.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

In any aspect or embodiment herein of methods of the present technology, administration may include but not be limited to, parenteral, intravenous, intramuscular, intradermal, intraperitoneal, intratracheal, subcutaneous, oral, intranasal/respiratory (e.g., inhalation), transdermal (topical), sublingual, ocular, vaginal, rectal, or transmucosal administration.

### EXAMPLES

The examples herein are provided to illustrate advantages of the present technology and to further assist a person of ordinary skill in the art with preparing and/or using the compounds of the present technology. The examples herein are also presented in order to more fully illustrate the preferred aspects of the present technology. The examples should in no way be construed as limiting the scope of the present technology. The examples can include or incorporate any of the variations, aspects, or embodiments of the present technology described above. The variations, aspects, or embodiments described above may also further each include or incorporate the variations of any or all other variations, aspects, or embodiments of the present technology.

### Experimental Materials and Methods

General. Unless otherwise stated, all solvents and reagents were obtained from Sigma-Aldrich or Fisher Scientific and were used without further purification. Chlorin-containing NHS ester (ChL-NHS) was prepared according to Gonzales et al., “Facile synthesis of chlorin bioconjugates by a series of click reactions,” Chem. Commun. 2017, 53, 3773-3776 (2017); a structural representation of ChL-NHS is provided below:

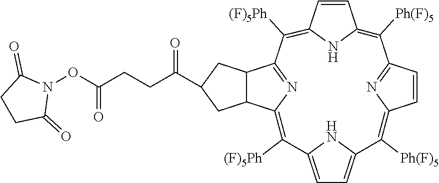

Anti-Na_{V}1.7 antibody [N68/6] was obtained from Abcam (ab85015). Water (>18.2 MΩcm⁻¹ at 25° C.) was obtained from an Alpha-Q Ultrapure water system (Millipore). Acetonitrile (AcN) and water (H₂O) were of high-performance liquid chromatography (HPLC) grade and of Liquid Chromatography Mass Spectroscopy (LCMS) grade, respectively. Phosphate Buffered Saline (PBS) without Ca²⁺ or Mg²⁺ was obtained from the Media Preparation Facility at Memorial Sloan Kettering Cancer Center and used for all in vivo injections. Reverse-phase (RP) HPLC purifications were performed on a Shimadzu HPLC system equipped with a DGU-20A degasser, SPD-M20A UV detector, LC-20AB pump system, and a CBM-20A communication BUS module using RP-HPLC columns (Atlantis T3 C18, 5 μm, 4.6×250 mm, P/N: 186003748). Electrospray ionization mass spectroscopy (ESI-MS) spectra was recorded with a Waters Aquity UPLC (Milford, Calif.) with an electrospray ionization SQ detector. Cherenkov imaging and Epifluorescence imaging were performed on an IVIS Spectrum (PerkinElmer). The radioactivity of organs for biodistribution was counted with a WIZARD² automated 7-counter from PerkinElmer and the histology slides of 10 μm of Human vagus nerves were obtained as a donation from the Fusion Solutions Bioskills Laboratory autopsy program. Confocal microscopy images were captured using a Leica SP8 inverted-stand confocal microscope equipped a tunable white light laser that ranges from 470-670 nm. The microscope is also equipped with a 405 nm diode for detection of Hoechst 33342, an argon laser (with 476 nm, 488 nm, 496 nm and 514 nm laser line) and a 610 nm laser for NIR imaging coupled with avalanche photo diode detectors (APDs) which were used for detection of Hsp1a-ChL.

Synthesis of sHsp1a. sHsp1a was synthesized using standard FMOC chemistry on a Symphony peptide synthesizer (Gyros Protein Technologies, Tucson, Ariz., USA), as previously described (Agwa et al., Biochim. Biophys. Acta 2017, 1859, 835-844). C-terminal amidation was achieved using a Rink-amide resin at a scale of 0.125 mmol. Simultaneous release from the resin and removal of side chain protecting groups occurred in a solution containing TFA/triisopropylsilane (TIPS)/water (48:1:1) (v/v/v) for 2.5 h. Crude sHsp1a was triturated in chilled diethyl ether, and then the precipitated peptide precipitate was dissolved in solvent A/B (45% v/v acetonitrile, 0.05% v/v TFA), lyophilized and prepurified using C18 RP-HPLC. The peptide was eluted using a linear gradient of 10-60% solvent B (90% v/v acetonitrile; 0.05% v/v TFA) over 50 min using a flow rate of 50 mL·min⁻¹. Fractions were collected and analyzed using electrospray ionization-mass spectroscopy (ESI-MS) and fractions of interest were pooled, lyophilized and stored at −20° C. sHsp1a (0.1 mg/mL) was oxidized for 16 h at room temperature in a buffer containing 2 M Urea, 0.1 M Tris pH 8, 0.15 mM reduced glutathione and 0.3 mM oxidized glutathione. Oxidation was quenched by acidification to pH 3, before the peptide was filtered and purified using preparatory and semi-preparatory RP-HPLC as previously described (Agwa et al., J. Biol. Chem. 2018, 293(23), 9041-9052). A single sharp peak was obtained from the final analytical RP-HPLC purification and 96% purity was achieved as calculated from area under the curve. LC-ESI-MS (ES+), m z calculated for [C₁₄₈H₂₂₀N₄₀O₄₀S₆] 3389.52, [C₁₄₈H₂₂₀N₄₀O₄₀S₆+2H]²⁺ 1696.76, found [M+2H]²⁺ 1696.00, [C₁₄₈H₂₂₀N₄₀O₄₀S₆+3H]³⁺ 1130.84, found [M+3H]3+1131.05, [C₁₄₈H₂₂₀N₄₀O₄₀S₆+4H]⁴⁺ 848.38, found [M+4H]4+848.55, [C₁₄₈H₂₂₀N₄₀O₄₀S₆+5H]⁵⁺ 678.90, found [M+5H]⁵+679.05.

Synthesis of Hsp1a-ChL. Hsp1a-ChL was synthesized using Hsp1a peptide (0.37 mM, 250 μg in 200 μL of ACN) in a solution of Na₂CO₃ (1M, 40 μL). ChL-NHS (50 μL of a 0.82 mM solution) was dissolved in ACN and added dropwise to the reaction mixture, which was allowed to react for 10 min. The product, Hsp1a-ChL, was purified using RP-HPLC. The excess solvent was removed in vacuo affording Hsp1a-ChL as a green powder (80 μg, 24% isolated yield). The final analytical RP-HPLC purification showed 96% purity. LC-ESI-MS (ES+), m/z calculated for [C₁₉₈H₂₃₆F₂₀N₄₆O₄₁S₆] 4485.58, [C₁₉₈H₂₃₆F₂₀N₄₆O₄₁S₆+3H]³⁺ 1496.19, found [M+3H]³⁺ 1497.23, [C₁₉₈H₂₃₆F₂₀N₄₆O₄₁S₆+4H]⁴⁺ 1122.40, found [M+4H]⁴⁺ 1123.03.

Synthesis of [⁴Cu]Cu-ChL-Hsp1a. ⁶⁴Cu was produced at the Mallinckrodt Institute of Radiology (Washington University) on an 19 MeV-beam energy cyclotron (Advanced Cyclotron Systems Inc. British Columbia, Canada) via the ⁶⁴Ni(p,n)⁶⁴Cu nuclear reaction and it was purified in accordance with Kozempel, J., et al. “A novel method for n.c.a. ⁶⁴Cu production by the ⁶⁴Zn(d, 2p)⁶⁴Cu reaction and dual ion-exchange column chromatography,” Radiochim. Acta 2009, 95, 75-80 to yield ⁶⁴Cu with a specific activity of 298-1830 mCi/μg (978 mCi/μg avg.). This eluate was directly used for ⁶⁴Cu-labelling experiments. Activity measurements were made using a Capintec CRC-15R Dose Calibrator (Capintec, Ramsey, N.J.).

Accordingly, [⁶⁴Cu]CuCl₂ (40-50 MBq) was added to a solution of 0.9 mL ammonium acetate buffer (0.1 M, pH=6.0) containing 100 μL of Hsp1a-ChL (1 mg mL⁻¹ in DMSO). After incubating the solution for 30 min at 37° C., the reaction mixture was then diluted with 1 mL of water and trapped on a Sep-Pack® Light tC18 cartridge (preconditioned with 10 mL ethanol and 10 mL water). The cartridge was washed with 2.5 mL of water and the labelled compound was eluted with 200 μL of a pure ethanol solution. Then, [⁶⁴Cu]Cu-ChL-Hsp1a was diluted with saline and it was used for in vivo injections. For blocked experiments, unlabeled Hsp1a-ChL (Hsp1a, 204 μM, 21 nmol in 100 μL PBS) was mixed in the aforementioned solution and it was used for injection. [⁶⁴Cu]Cu-ChL-Hsp1a gave radiochemical conversions >70%, and the product was obtained in >95% radiochemical purity, and in 40-50% isolated RCY (n.d.c.), which corresponded to maximum molar activities of 1-2 MBq nmol-1. The synthesis and isolation were performed within about 60 min.

Synthesis of [^{nat.}Cu]Cu-ChL-Hsp1a. Chelation of Hsp1a-ChL to “cold” copper was carried out similar to synthesis of [⁶⁴Cu]Cu-ChL-Hsp1a to yield [^{nat.}Cu]Cu-ChL-Hsp1a. Absorption and emission for [^{nat.}Cu]Cu-ChL-Hsp1a were determined as well. The isolated product was confirmed by instant thin layer chromatography (iTLC) and RP-HPLC. The retention time for Hsp1a-ChL (observed at 280 nm) and [⁶⁴Cu]Cu-ChL-Hsp1a was 21 min in both cases.

Synthesis of Hsp1a_Pra0. Similar to sHsp1a, Hsp1a_Pra0 was synthesized using standard FMOC chemistry on a Symphony peptide synthesizer (Gyros Protein Technologies, Tucson, Ariz., USA), as previously described (Agwa et al., Biochim. Biophys. Acta 2017, 1859, 835-844). The insertion of glycine carrying a propargyl group was possible by the use of Fmoc-L-2-propargylglycine in the synthesis. C-terminal amidation was achieved using a Rink-amide resin at a scale of 0.125 mmol. Simultaneous release from the resin and removal of side chain protecting groups occurred in a solution containing TFA/triisopropylsilane (TIPS)/water (48:1:1) (v/v/v) for 2.5 h. Crude Hsp1a_Pra0 was triturated in chilled diethyl ether, and then the precipitated peptide precipitate was dissolved in solvent A/B (45% v/v acetonitrile, 0.05% v/v TFA), lyophilized and prepurified using C18 RP-HPLC. The peptide was eluted using a linear gradient of 10-60% solvent B (90% v/v acetonitrile; 0.05% v/v TFA) over 50 min using a flow rate of 50 mL·min⁻¹. Fractions were collected and analyzed using electrospray ionization-mass spectroscopy (ESI-MS) and fractions of interest were pooled, lyophilized and stored at −20° C. sHsp1a (0.1 mg/mL) was oxidized for 16 h at room temperature in a buffer containing 2 M Urea, 0.1 M Tris pH 8, 0.15 mM reduced glutathione and 0.3 mM oxidized glutathione. Oxidation was quenched by acidification to pH 3, before the peptide was filtered and purified using preparatory and semi-preparatory RP-HPLC as previously described (Agwa et al., J. Biol. Chem. 2018, 293(23), 9041-9052). A single sharp peak was obtained from the final analytical RP-HPLC purification and 96% purity was achieved as calculated from area under the curve. LC-ESI-MS (ES+), m z calculated for [C₁₅₃H₂₂₆N₄₂O₄OS₆] 3483.53, [C₁₅₃H₂₂₆N₄₂O₄₀S₆+2H]²⁺ 1742.77, found [M+2H]²+1743.80, [C₁₅₃H₂₂₆N₄₂O₄₀S₆+3H]²⁺ 1162.18, found [M+3H]3+1163.38, [C₁₅₃H₂₂₆N₄₂O₄₀S₆+4H]⁴⁺ 871.88, found [M+4H]4+ 872.75.

Synthesis of Hsp1a_Pra4 and Hsp1a_Pra26. Hsp1a_Pra4 and Hsp1a_Pra26 were synthesized in a similar fashion as Hsp1a_Pra0.

Table 1 illustrates the sequence of Hsp1a_Pra0, Hsp1a_Pra4, and Hsp1a_Pra26 alongside Hsp1a.

TABLE 1
Sequence of Hsp1a, Hsp1a_Pra0,
Hsp1a_Pra4, and Hsp1a_Pra26.

| Compound | Amino Acid Sequence |
|---|---|
| Hsp1a | YCQKFLWTCDSERPCCEGLVCRLWCKIN-NH₂ |
| | (SEQ ID NO: 2) |
| Hsp1a_ | PraYCQKFLWTCDSERPCCEGLVCRLWCKIN-NH₂ |
| Pra0 | (SEQ ID NO: 3) |
| Hsp1a_ | YCQPraFLWTCDSERPCCEGLVCRLWCKIN-NH₂ |
| Pra4 | (SEQ ID NO: 4) |
| Hsp1a_ | YCQKFLWTCDSERPCCEGLVCRLWCPraIN-NH₂ |
| Pra26 | (SEQ ID NO: 5) |

Synthesis of IR800_azide, Janelia669 azide, BODIPY665_azide, Cy7.5_azide, and DY684_azide. In general, IR800_azide, Janelia669_azide, BODIPY665_azide, Cy7.5_azide, and DY684_azide were synthesized by diluting 2 mg (9.2 μmol) 11-azido-3,6,9-trioxaundecan-1-amine in dichloromethane (0.5 mL) followed by adding 2 mg of corresponding IR800, Janelia669, BODIPY665, Cy7.5 and DY684-NHS esters (1.8 μmol, 2.9 μmol, 3.1 μmol, 1.8 μmol and 2.4 μmol respectively, all compounds dissolved in 100 L of acetonitrile just before addition) inside an amber vial. The reaction mixtures were stirred at room temperature in the dark for at least 1 h. The solvent was removed under reduced pressure and the individual crude reaction mixtures were subjected to HPLC purification, yielding 0.5 mg in average. The final analytical RP-HPLC showed 95-98% purity. The structures of IR800_azide, Janelia669_azide, BODIPY665_azide, Cy7.5_azide, and DY684_azide are provided below:

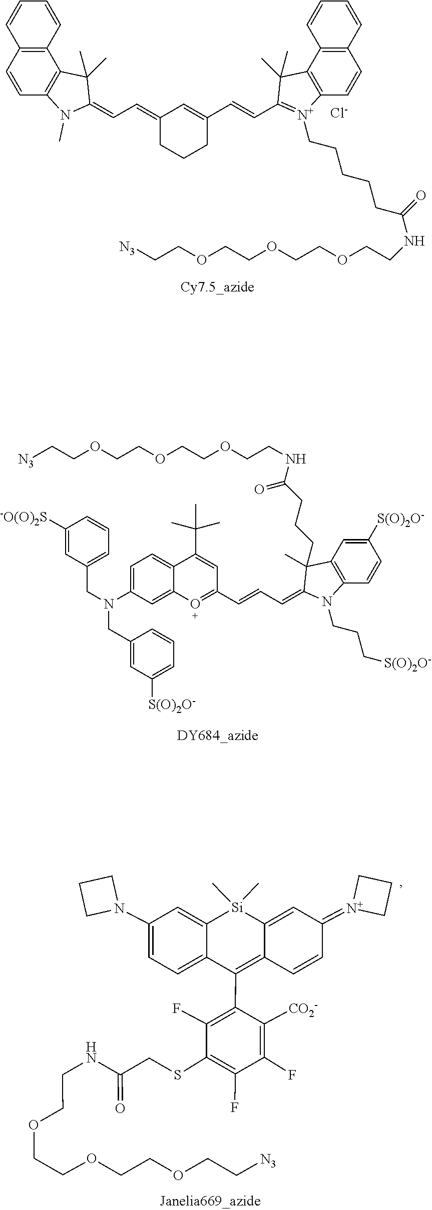

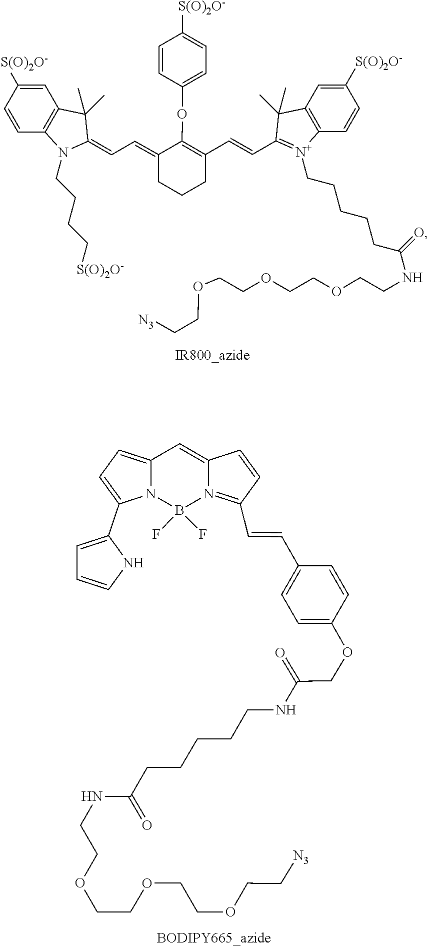

Synthesis of Hsp1a_Pra0 IR800. Hsp1a_Pra0_IR800 was synthesized using Hsp1a_Pra0 peptide (0.71 mM, 250 μg in 100 μL of H₂O), which was diluted in 100 μL of a 25-mM Tris-buffered aqueous solution. To this reaction mixture, 20 μL of a 50-mM solution of L-ascorbic acid in H₂O and 20 μL of a 50-mM aqueous CuSO₄ solution in H₂O, were added. Immediately, to the same reaction, 104 μg (87 nmol) of IR-800 azide in 50 μL of 50:50 (ACN/H₂O) solution was added, and the reaction mixture was stirred at room temperature in the dark for 4 h. The crude mixture was subjected to HPLC purification, yielding 88 μg (19 nmol; 26%). The final analytical RP-HPLC purification showed 96% purity. LC-ESI-MS (ES+), m/z calculated for Hsp1a_Pra0_IR800, [C₂₀₇H₂₉₃N₄₉O₅₇S₁₀]4682.87, [C₂₀₇H₂₉₃N₄₉O₅₇S₁₀+3H]³⁺ 1563.09, found [M+3H]³⁺ 1564.10, [C₂₀₇H₂₉₃N₄₉O₅₇S₁₀+4H]⁴⁺ 1172.00, found [M+4H]⁴⁺ 1173.20, [C₂O₇H₂₉₃N₄₈O₅₇S₁₀+5H]⁵⁺ 938.16, found [M+5H]⁵⁺ 939.00.

Synthesis of Hsp1a_Pra0 Janelia669. Hsp1a_Pra0_Janelia669 was synthesized using Hsp1a_Pra0 peptide (0.71 mM, 250 μg in 100 μL of H₂O), which was diluted in 100 μL of a 25-mM Tris-buffered aqueous solution. To this reaction mixture, 20 μL of a 50-mM solution of L-ascorbic acid in H₂O and 20 μL of a 50-mM aqueous CuSO₄ solution in H₂O, were added. Immediately, to the same reaction, 69 μg (87 nmol) of Janelia669 azide in 50 μL of 50:50 (ACN/H₂O) solution was added, and the reaction mixture was stirred at room temperature in the dark for 4 h. The crude mixture was subjected to HPLC purification, yielding 95 μg (22 nmol; 31%). The final analytical RP-HPLC purification showed 95% purity. LC-ESI-MS (ES+), m/z calculated for Hsp1a_Pra0_Janelia669, [C₁₉₁H₂₆₉F₃N₄O₄₆S₇Si] 4682.87, [C₁₉₁H₂₆₉F₃N₄₉O₄S₇Si+3H]³⁺ 1428.00, found [M+3H]³⁺ 1429.14, [C₁₉₁H₂₆₉F₃N₄O₄₆S₇Si+4H]⁴⁺ 1071.07, found [M+4H]⁴⁺ 1072.10, [C₁₉₁H₂₆₉F₃N₄O₄₆S₇Si+5H]⁵⁺ 965.30, found [M+5H]⁵⁺ 965.50.

Synthesis of Hsp1a_Pra0 BODIPY665. Hsp1a_Pra0_BODIPY665 was synthesized using Hsp1a_Pra0 peptide (0.71 mM, 250 μg in 100 μL of H₂O), which was diluted in 100 μL of a 25-mM Tris-buffered aqueous solution. To this reaction mixture, 20 μL of a 50-mM solution of L-ascorbic acid in H₂O and 20 μL of a 50-mM aqueous CuSO₄ solution in H₂O, were added. Immediately, to the same reaction, 65 μg (87 nmol) of BODIPY665 azide in 50 μL of 50:50 (ACN/H₂O) solution was added, and the reaction mixture was stirred at room temperature in the dark for 4 h. The crude mixture was subjected to HPLC purification, yielding 70 μg (17 nmol; 23%). The final analytical RP-HPLC purification showed 96% purity. LC-ESI-MS (ES+), m/z calculated for Hsp1a_Pra0_BODIPY665, [C₁₉₀H₂₇₁BF₂N₅₀O₄S₆] 4229.88, [C₁₉₀H₂₇₁BF₂N₅₀O₄S₆+3H]³⁺ 1411.00, found [M+3H]³⁺ 1412.22, [C₁₉₀H₂₇₁BF₂N₅₀O₄₆S₆+4H]⁴⁺ 1059.00, found [M+4H]⁴⁺ 1060.10.

Synthesis of Hsp1a_Pra0 Cy7.5. Hsp1a_Pra0_Cy7.5 was synthesized using Hsp1a_Pra0 peptide (0.71 mM, 250 μg in 100 μL of H₂O), which was diluted in 100 μL of a 25-mM Tris-buffered aqueous solution. To this reaction mixture, 20 μL of a 50-mM solution of L-ascorbic acid in H₂O and 20 μL of a 50-mM aqueous CuSO₄ solution in H₂O, were added. Immediately, to the same reaction, 73 μg (86 nmol) of Cy7.5 azide in 50 μL of 50:50 (ACN/H₂O) solution was added, and the reaction mixture was stirred at room temperature in the dark for 4 h. The crude mixture was subjected to HPLC purification, yielding 90 μg (21 nmol; 29%). The final analytical RP-HPLC purification showed 96% purity. LC-ESI-MS (ES+), m/z calculated for Hsp1a_Pra0_Cy7.5, [C₂₀₆H₂₉₁N₄₈O₄₄S₆] 4333.03, [C₂₀₆H₂₉₁N₄₉O₄₄S₆+3H]³⁺ 1445.24, found [M+3H]³⁺ 1446.30, [C₂₀₆H₂₉₁N₄₉O₄₄S₆+4H]⁴⁺ 1084.20, found [M+4H]⁴⁺ 1085.05, [C₂₀₆H₂₉₁N₄₉O₄₄S₆+5H]⁵⁺ 867.60, found [M+5H]5+869.00.

Synthesis of Hsp1a_Pra0 DY684. Hsp1a_Pra0_DY684 was synthesized using Hsp1a_Pra0 peptide (0.71 mM, 250 μg in 100 μL of H₂O), which was diluted in 100 μL of a 25-mM Tris-buffered aqueous solution. To this reaction mixture, 20 μL of a 50-mM solution of L-ascorbic acid in H₂O and 20 μL of a 50-mM aqueous CuSO₄ solution in H₂O, were added. Immediately, to the same reaction, 103 μg (86 nmol) of DY684 azide in 50 μL of 50:50 (ACN/H₂O) solution was added, and the reaction mixture was stirred at room temperature in the dark for 4 h. The crude mixture was subjected to HPLC purification, yielding 75 μg (16 nmol; 22%). The final analytical RP-HPLC purification showed 95% purity. LC-ESI-MS (ES+), m/z calculated for Hsp1a_Pra0_DY684, [C₂₀₇H₂₈₉N₄₉O₅₇S₁₀]4682.87, [C₂₀₇H₂₈₉N₄₉O₅₇S₁₀+3H]³⁺ 1561.15, found [M+3H]³⁺ 1562.05, [C₂₀₇H₂₈₉N₄₉O₅₇S₁₀+4H]⁴⁺ 1171.00, found [M+4H]⁴⁺ 1171.12, [C₂₀₇H₂₈₉N₄₈O₅₇S₁₀+5H]⁵+938.16, found [M+5H]⁵+937.08.

Animal Model. Female athymic nude mice (4-8 week-old, athymic-Nude (outbred) (Stock #:088; Envigo, USA) were allowed to acclimatize at the Memorial Sloan Kettering Cancer Center (MSK) vivarium for 1 week with ad libitum food and water prior to the experimental procedure. For epifluorescence and Cherenkov imaging experiments, animals were sacrificed 30 min post-tail vein injection (e.g., of either Hsp1a-ChL, [⁶⁴Cu]Cu-ChL-Hsp1a, Hsp1a/[⁶⁴Cu]Cu-ChL-Hsp1a or PBS). All animal experiments were performed in accordance with institutional guidelines and approved by the IACUC of MSK, following NIH guidelines for animal welfare.

Human Tissue. Human nerves (vagus nerves, n=6) were a donation from the Fusion Solutions Bioskills Laboratory, Long Island, N.Y. Nerves were sectioned at 10 μm thickness for staining with Hsp1a-ChL. In addition, the nerves were paraffin-embedded and formalin fixed and sectioned at 10 m thickness for H&E and immunohistochemical detection experiments.

Immunohistochemistry. Na_{V}1.7 in human vagus nerves and mouse sciatic nerves was detected using immunohistochemical (IHC) staining techniques, which were performed at the Molecular Cytology Core Facility of MSK using the Discovery XT processor (Ventana Medical System, Tucson, Ariz.). Anti-Na_{V}1.7 antibody [N68/6] (Abcam ab85015) specifically bound to both human and mouse Na_{V}1.7 (0.5 μg/mL). Paraffin-embedded formalin-fixed 10 m sections were deparaffinized with EZPrep buffer. For IHC detection, a 3,3′-diaminobenzidine (DAB) detection kit (Ventana Medical Systems, Tucson, Ariz.) was used according to the manufacturer's instructions. Sections were counterstained with H&E and coverslipped with Permount (Fisher Scientific, Pittsburgh, Pa.).

Confocal Microscopy. Staining with Hsp1a-ChL was performed in both mouse sciatic nerves and human vagus nerves. For mice, 10 μm cryosections of OCT-embedded sciatic nerve tissues were used from mice previously injected with Hsp1a-ChL (1 nmol, 10 μM of Hsp1a-ChL in 100 μL PBS), Hsp1a/Hsp1a-ChL (Hsp1a-ChL, 10 μM, 1 nmol and Hsp1a, 204 μM, 21 nmol in 100 μL PBS) or PBS. Tissues were counterstained with 33342 (blue, 20 μM, 1 nmol in 50 μL of PBS) up to 90 min post-mortem and placed directly on a microscope slide for imaging. For human tissue, 10 μm cryosections of vagus nerve were immersed in Hsp1a-ChL (3 nmol, 30 μM of Hsp1a-ChL in 100 μL PBS), Hsp1a/Hsp1a-ChL (Hsp1a-ChL, 30 μM, 3 nmol and Hsp1a, 90 μM, 9 nmol in 100 μL PBS) or PBS. Tissues were incubated with Hoechst 33342 (blue, 20 μM, 1 nmol in 50 μL of PBS) to counterstain nuclei. For human vagus nerve tissue, to obtain fluorescence signal, the specimen were incubated for at least 2 min, followed by one PBS wash cycle. For the block experiment, nerve tissue was first immersed in Hsp1a solution for at least 5 min, followed by immersion in Hsp1a-ChL for at least 2 min, followed by one PBS wash cycle.

Epifluorescence Imaging of Hsp1a-ChL. Animals were intravenously injected with Hsp1a-ChL (1 nmol, 10 μM of Hsp1a-ChL in 100 μL PBS, n=6). To assess the specificity of the Hsp1a-ChL accumulation, we injected a combination of Hsp1a and Hsp1a-ChL (Hsp1a-ChL, 10 μM, 1 nmol and Hsp1a, 204 μM, 21 nmol in 100 μL PBS, n=6) or PBS (n=9). Animals were sacrificed 30 min post-injection and epifluorescence images obtained. Epifluorescence images of right sciatic nerves (RSN) and left sciatic nerves (LSN) were obtained first in situ and then ex vivo, together with excised muscle, heart, kidney, liver and brain tissue using an IVIS Spectrum (PerkinElmer) with a predefined filterset (excitation=600/20 nm, emission=650-670 nm). Autofluorescence was removed through spectral unmixing. Semiquantitative analysis of the Hsp1a-ChL signal was conducted by measuring the average radiant efficiency (in units of unit [p/s/cm²/sr]/[μW/cm²]) in regions of interest (ROIs) that were placed on all resected organs under white light guidance.

Epifluorescence Imaging of Hsp1a_Pra0 IR800, Hsp1a_Pra0 Janelia669, Hsp1a_Pra0 BODIPY665, Hsp1a_Pra0 Cy7.5, and Hsp1a_Pra0_DY684. In general and for each of Hsp1a_Pra0_IR800, Hsp1a_Pra0_Janelia669, Hsp1a_Pra0_BODIPY665, Hsp1a_Pra0_Cy7.5, and Hsp1a_Pra0_DY684 (collectively referred to as “Hsp1a_Pra0_NIR”) peptide used, animals were intravenously injected with the corresponding Hsp1a_Pra0_NIR (1 nmol, 10 μM of Hsp1a_Pra0_NIR in 100 μL PBS, n=6). To assess the specificity of the Hsp1a_Pra0 accumulation, we injected a combination of Hsp1a_Pra0 and Hsp1a_Pra0_NIR (Hsp1a_Pra0_NIR, 10 μM, 1 nmol and Hsp1a_Pra0, 204 μM, 21 nmol in 100 μL PBS, n=6) or PBS (n=6). Animals were sacrificed 30 min post-injection and epifluorescence images obtained. Epifluorescence images of right sciatic nerves (RSN) and left sciatic nerves (LSN) tissues were obtained in situ using an IVIS Spectrum (PerkinElmer) with a corresponding predefined filterset. Autofluorescence was removed through spectral unmixing. Semiquantitative analysis of the Hsp1a_Pra0_NIR signal was conducted by measuring the average radiant efficiency (in units of [p/s/cm²/sr]/[μW/cm²]) in regions of interest (ROIs) that were placed on all resected organs under white light guidance.

Cerenkov Luminescence Imaging. Animals were intravenously injected with [⁶⁴Cu]Cu-ChL-Hsp1a (3.5-4.2 MBq in 200 μL of PBS, n=9). To assess the specificity of the [⁶⁴Cu]Cu-ChL-Hsp1a accumulation, we injected a combination of Hsp1a and [⁶⁴Cu]Cu-ChL-Hsp1a, 3.5-4.2 MBq and Hsp1a, 204 μM, 21 nmol in 200 μL PBS, n=6) or PBS (n=9). Animals were sacrificed 30 min post-injection and Cerenkov images obtained. Cerenkov Luminescence images of right sciatic nerves and left sciatic nerve were obtained first in situ with a surgical cut to expose the sciatic nerves, and then of the resected RSN and LSN. Images of the excised RSN, LSN, muscle, heart, kidney, liver and brain were also obtained, and Cherenkov luminescence imaged with an IVIS Spectrum (PerkinElmer). Semiquantitative analysis of the [⁶⁴Cu]Cu-ChL-Hsp1a signal was conducted by measuring the radiant efficiency in regions of interest (ROIs) that were placed on all resected organs under white light guidance. Moreover, the radioactivity of organs for biodistribution was counted with a WIZARD² automated 7-counter from PerkinElmer.

Statistical Analysis. Unless otherwise stated, data points represent mean values and error bars represent the standard deviation of biological replicates (mean±SEM). All p-values were calculated using a Student's unpaired t-test. Statistical significance was considered for p-values<0.05 and as follows: ns=not significant, *p<0.05, **p<0.01, ***p<0.001. Mann-Whitney tests were used for analysis of the unpaired samples (e.g. vital signs in mice injected with Hsp1a and mice from the control group) and the Wilcoxon test was used for analysis of paired samples (e.g. vital signs from same mouse before and after injection). Statistical significance was determined with alpha=0.05. Analysis and figures were prepared in GraphPad Prism 8.

### Example 1: Results for Hsp1a-ChL, [^{nat}Cu]Cu-ChL-Hsp1a, and [⁶⁴Cu]Cu-ChL-Hsp1a

Pharmacokinetics of Hsp1a-ChL and [^{nat}Cu]Cu-ChL-Hsp1a.

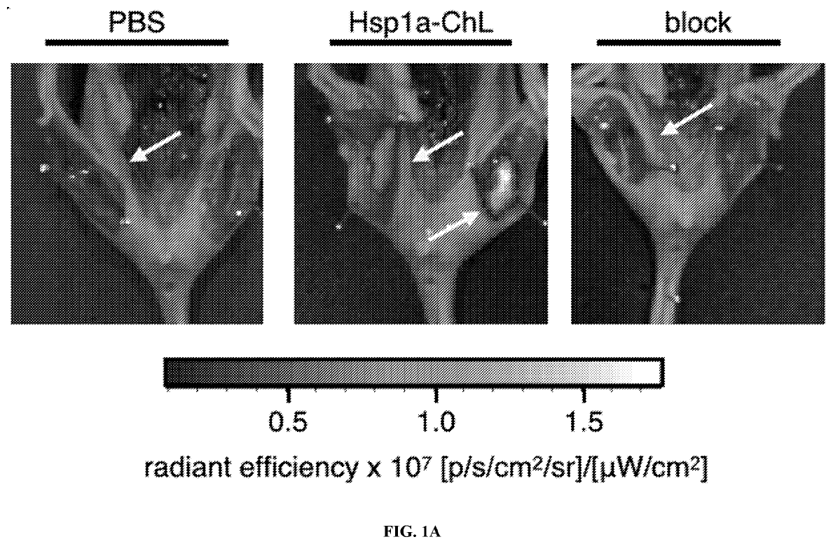

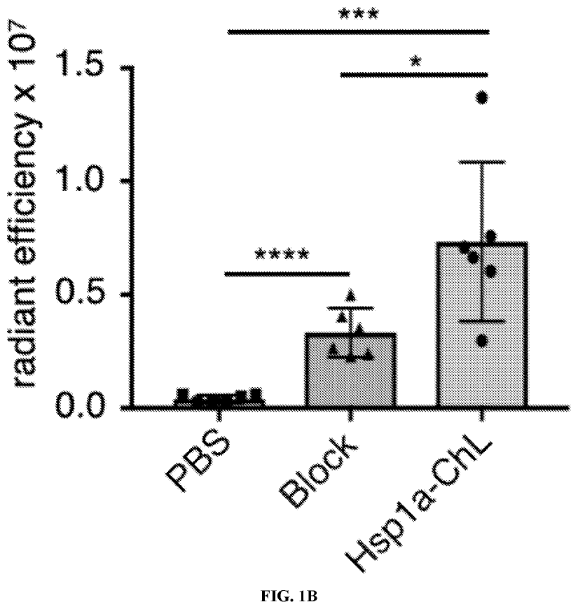

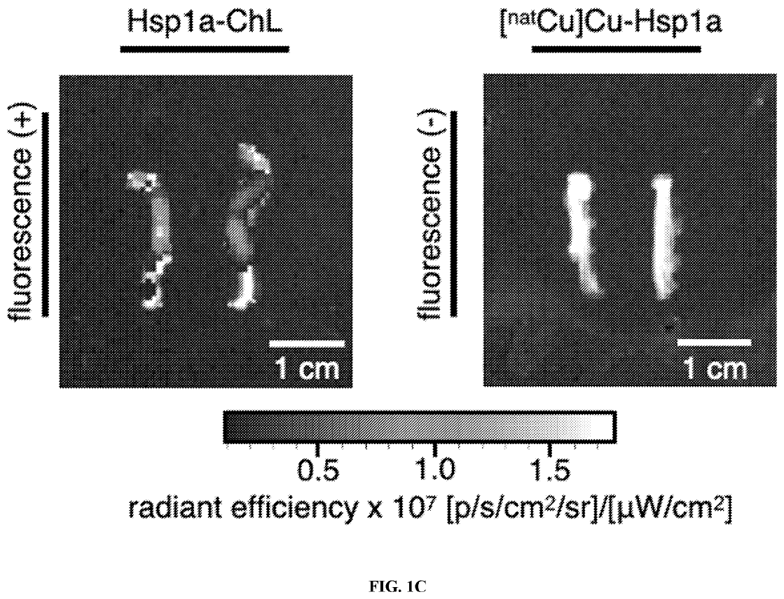

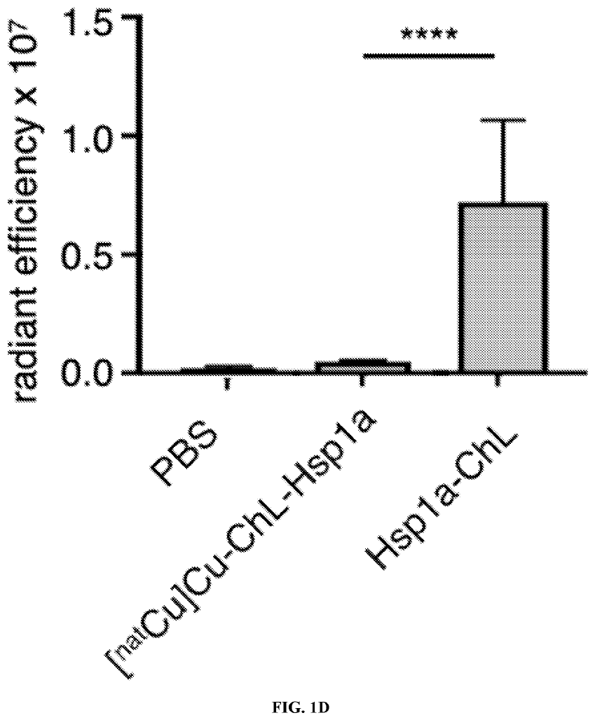

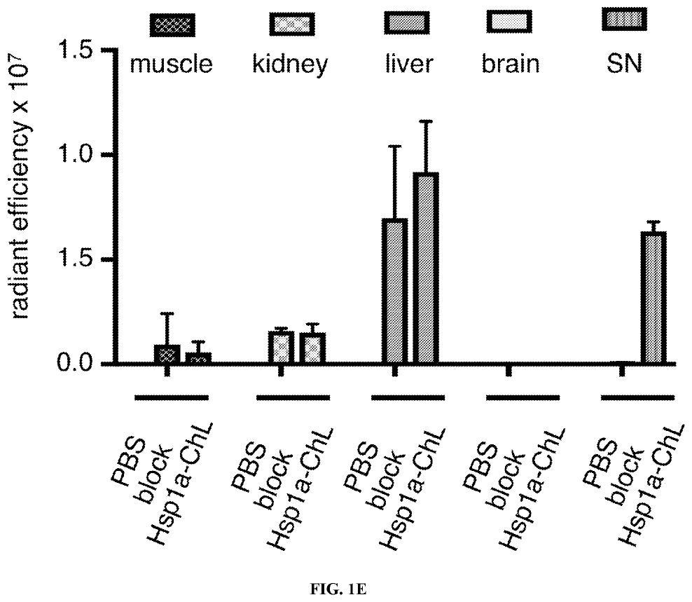

Under mesoscopic imaging conditions, we observed rapid and selective accumulation of Hsp1a-ChL within the peripheral nerves of mice (FIGS. 1A-E). For Hsp1a-ChL, mice were injected intravenously (1 nmol, 10 μM of Hsp1a-ChL in 100 μL PBS) with the fluorescent peptide alone or in combination with an excess of the unmodified Hsp1a peptide (Hsp1a-ChL, 10 μM, 1 nmol and Hsp1a, 21 nmol, 204 μM, 21 nmol in 100 μL PBS) and sacrificed 30 min after tail vein injection. The right and left sciatic nerves were then exposed and epifluorescence imaging performed using an IVIS Spectrum in vivo imaging system (ex: 600/20 nm; em: 650-670 nm). In mice receiving just the imaging agent, the sciatic nerves were clearly visible, whereas in mice receiving the imaging agent in combination with the unmodified peptide, uptake was significantly reduced (radiant efficiency: 0.7+0.4×10⁷ and 0.3+0.1×10⁷ for Hsp1a-ChL and co-injection (blocking), respectively, Student's unpaired t-test, P<0.0236, FIGS. 1A and 1B).

This was also corroborated under ex vivo imaging conditions, where high fluorescence uptake for the sciatic nerves of animals injected with Hsp1a-ChL was found. A mean radiant efficiency of 0.5+0.1×10⁷ was observed, whereas in animals receiving the imaging agent in combination with the unmodified peptide (21 nmol, 204 μM in 100 μL of PBS), a statistically significant 93-fold reduction in radiant efficiency was observed (0.005 0.001×10⁷ radiant efficiency, P=0.0012) (FIG. 1E). A trend towards higher fluorescence intensities in the livers of animals injected with the imaging agent only was observed (radiant efficiency: 0.92±0.14×10⁷ and 0.72±0.19×10⁷ for livers injected with Hsp1a-ChL and Hsp1a/Hsp1a-ChL, respectively). A similar trend was observed for the kidneys (radiant efficiency: 0.2±0.02×10⁷ and 0.7±0.2×10⁷ for injected with Hsp1a-ChL and Hsp1a/Hsp1a-ChL, respectively). No significant fluorescence was observed in any other organs, including the muscle, heart, kidney, spleen and brain when comparing animals injected with Hsp1a-ChL and PBS (FIG. 1E).

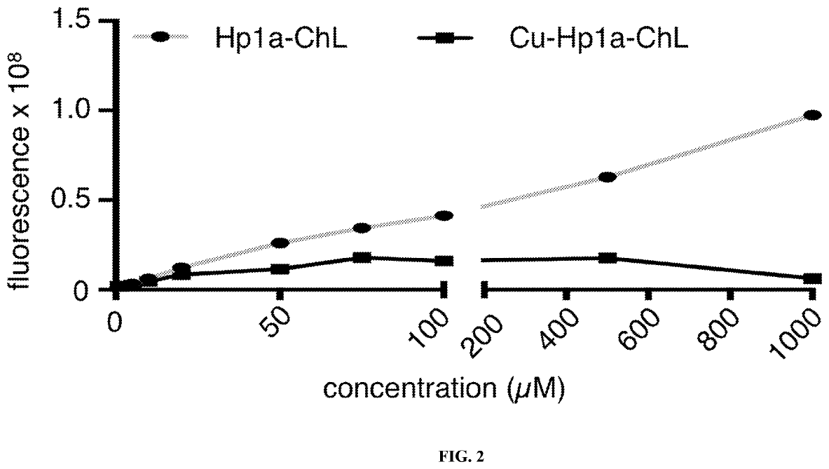

The fluorescence emission profiles of Hsp1a-ChL and [^{nat}Cu]Cu-ChL-Hsp1a were compared. FIG. 2 illustrates a clear dependence of emission upon Cu(II) complex formation, resulting in near-complete quenching after chelation. No significant fluorescence emissions were detected when animals were injected with the radiolabeled Hsp1a peptide (mean radiant efficiency ex vivo: 0.0008±0.000×10⁷) (FIGS. 1C-D). In this direction, fluorescence quenching is produced as an energy transfer process (intersystem crossing) from the excited electrons in the chlorin part to the metal center, for instant Cu(II) metal, an event that disturbs the fluorescence of the chlorin dye.

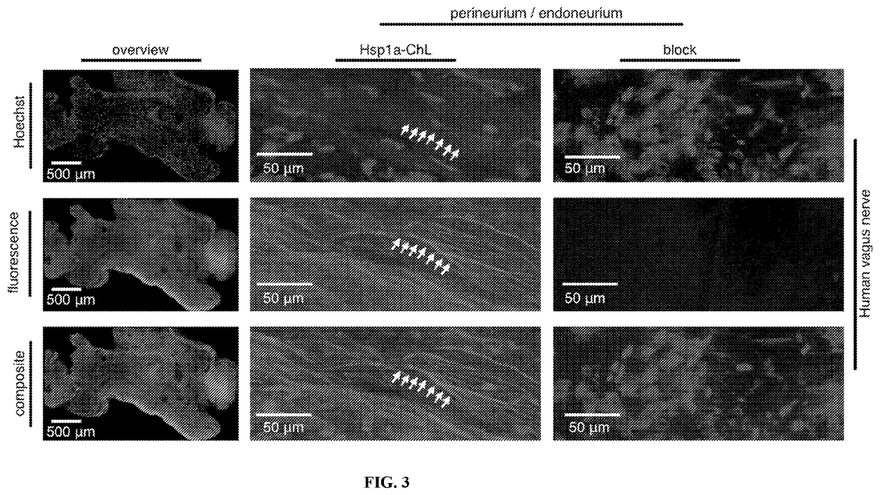

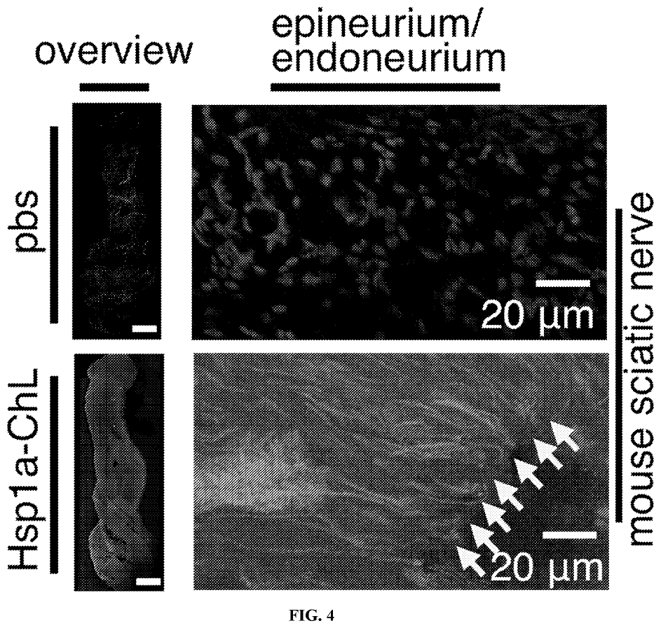

Histological Correlation and Validation of Hsp1a-ChL. Hsp1a imaging agents highlight nerve structures on a mesoscopic level and at cellular resolution. Consistent with this, experiments by the inventors have confirmed physiological similarities of human vagus and mouse sciatic nerves, we observed nerve patterns similar in both types of tissues. Specifically, for topical staining of human vagus nerves, the tissues were immersed in a solution of either Hsp1a-ChL (3 nmol, 30 μM of Hsp1a-ChL in 100 μL PBS), Hsp1a/Hsp1a-ChL (Hsp1a-ChL, 30 μM, 3 nmol and Hsp1a, 90 μM, 9 nmol in 100 μL PBS) or PBS. Additionally, tissues were incubated with Hoechst 33342 (blue, 20 μM, 1 nmol in 50 μL of PBS) to counterstain nuclei of Schwann cells (FIG. 3). Fluorescence signal showed venous patterns when nerves were sliced longitudinally consistent with nerve physiology and likely representing axons. These patterns were not observed when Hsp1a-ChL was used after incubation with Hsp1a. Moreover, histological analysis was performed on mouse sciatic nerves which were injected with Hsp1a-ChL (1 nmol, 10 μM in 100 μL of PBS) or block formulation (Hsp1a-ChL, 10 μM, 1 nmol and Hsp1a, 21 nmol, 204 μM, 21 nmol in 100 μL PBS) before their sciatic nerves were removed and sectioned for imaging. Mouse nerves showed similar staining patterns, corroborating the specificity of Hsp1a-ChL when staining human nerves (FIG. 4). Co-injection of Hsp1a prevented uptake of Hsp1a-ChL in sciatic nerves, indicating that the probe was specific and its target saturable.

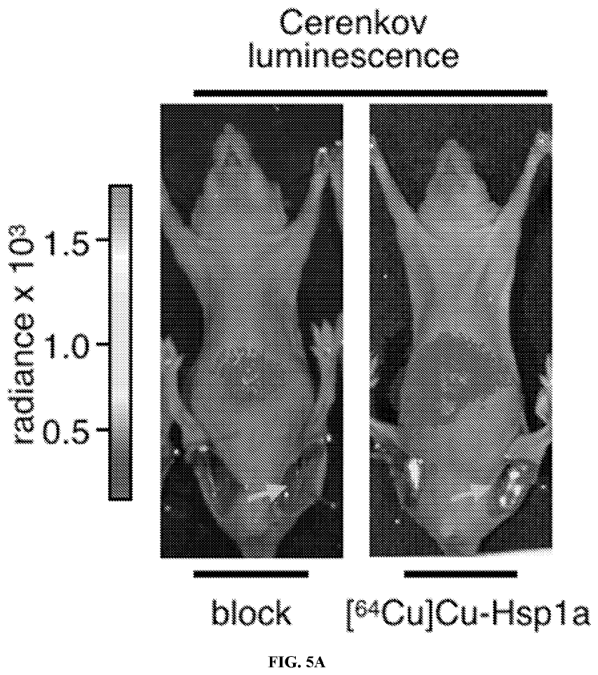

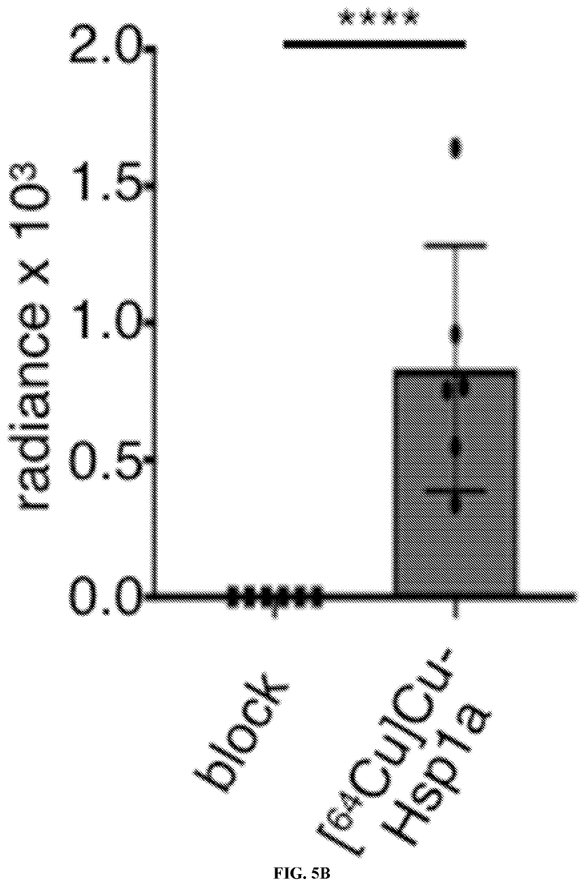

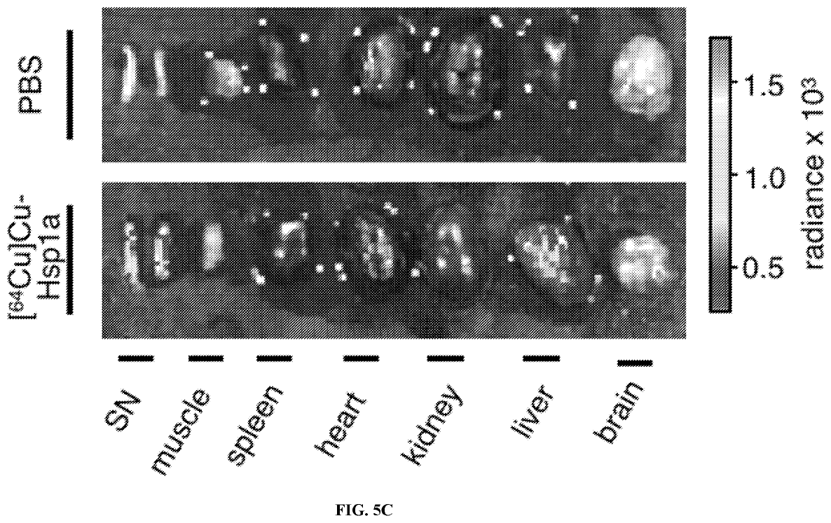

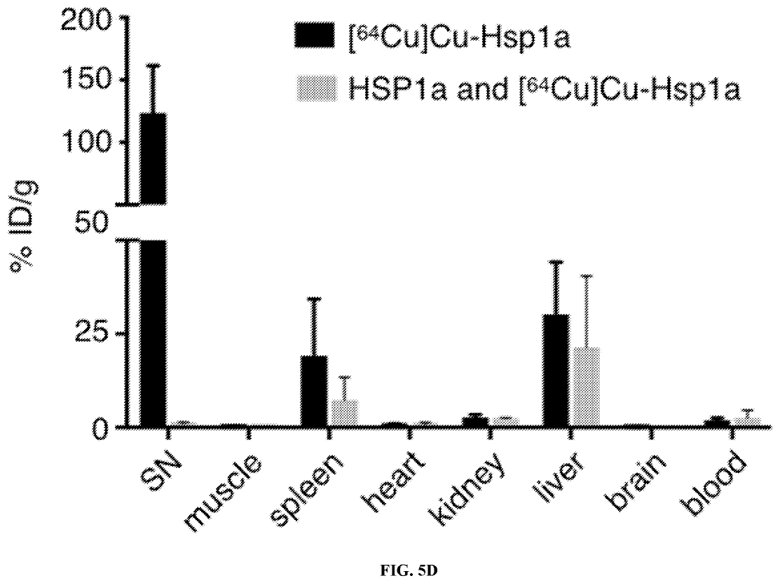

Pharmacokinetics of [⁴Cu]Cu-ChL-Hsp1a. For [⁶⁴Cu]Cu-ChL-Hsp1a, the uptake specificity in sciatic nerves was striking (FIGS. 5A-D). In mice receiving the imaging agent only (3.5-4.2 MBq in 200 μL of PBS), the sciatic nerves are clearly visible, with a mean radiant efficiency of 6830±2434 (FIGS. 5A-B), whereas mice receiving the imaging agent in combination with the unmodified peptide (21 nmol, 204 μM in 100 μL of PBS) had a statistically significant (Student's unpaired t-test, P<0.0001, 48-fold reduced 143±58 radiant efficiency in vivo. While it should be noted that quantitative assessment of Cerenkov Luminescence across organ systems is not feasible, the only site of off-target uptake both in vivo and ex vivo appears to be the liver (FIGS. 5C-D). No notable uptake was observed in other organs, including the muscle, kidney, heart and brain for animals injected with the combination of [⁶⁴Cu]Cu-ChL-Hsp1a and unmodified Hsp1a. Following this, acute bio-distribution studies were performed and radioactivity associated with each organ was expressed as a percentage of injected dose per gram of organ (% ID/g). This quantitative measure showed uptake in sciatic nerves, liver and spleen (FIG. 5D). The contrasting results of uptake in the spleen between CLI and gamma counter may be explained by the swallowing of the Cerenkov luminescence in dark, highly absorbing tissue.

### Example 2: Results for Hsp1a_Pra_IR800, Hsp1a_Pra0_Janelia669, Hsp1a_Pra0_BODIPY665, Hsp1a_Pra0 Cy7.5, and Hsp1a_Pra0_DY684

TABLE 2
Synthesized Hsp1a_Pra0 derivatives, analytics, and in vivo performance.

| | Isolated | | | | | | |
|---|---|---|---|---|---|---|---|
| | yield & | Peptide | | | | | Max |
| | retention | length | | | | Injected | fluorescent |
| | time | (# of | | | Observed | amount, | signal |
| | (RP- | amino | Fluorescence | MW | ion | total vol | [p/s/cm²/sr]/ |
| Peptide | HPLC) | acids) | peak | (kDa) | LC-MS | 100 μL | [μW/cm²] |
| Hsp1a_Pra0 | 96%, 20 | 29 | none | 3484 | [M + 2H]²⁺ | — | — |
| | min | | | | [M + 3H]³⁺ | | |
| Hsp1a_Pra0_IR800 | 94%, 21 | 29 | 800 nm | 4683 | [M + 2H]²⁺ | 1 nmol | 10⁹ |
| | min | | | | [M + 5H]⁵⁺ | | |
| Hsp1a_Pra0_Janelia669 | 95%, 24 | 29 | 680 nm | 4550 | [M + 2H]²⁺ | 1 nmol | 10⁸ |
| | min | | | | [M + 3H]³⁺ | | |
| Hsp1a_Pra0_BODIPY665 | 94%, 25 | 29 | 670 nm | 4230 | [M + 2H]²⁺ | 1 nmol | 10⁷ |
| | min | | | | [M + 3H]³⁺ | | |
| Hsp1a_Pra0_DY684 | 94%, 27 | 29 | 690 nm | 4679 | [M + 2H]²⁺ | 1 nmol | 10⁷ |
| | min | | | | [M + 3H]³⁺ | | |
| Hsp1a_Pra0_Cy7.5 | 96%, 28 | 29 | 790 nm | 4333 | [M + 2H]²⁺ | 1 nmol | 10⁵ |
| | min | | | | [M + 3H]³⁺ | | |

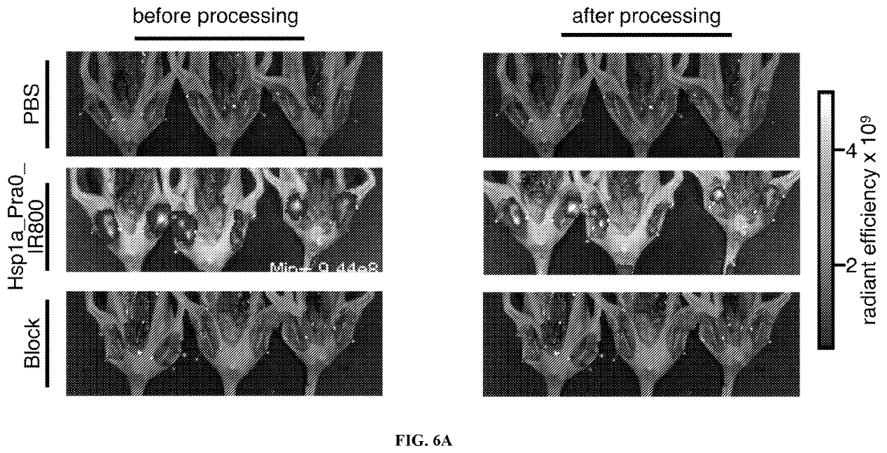

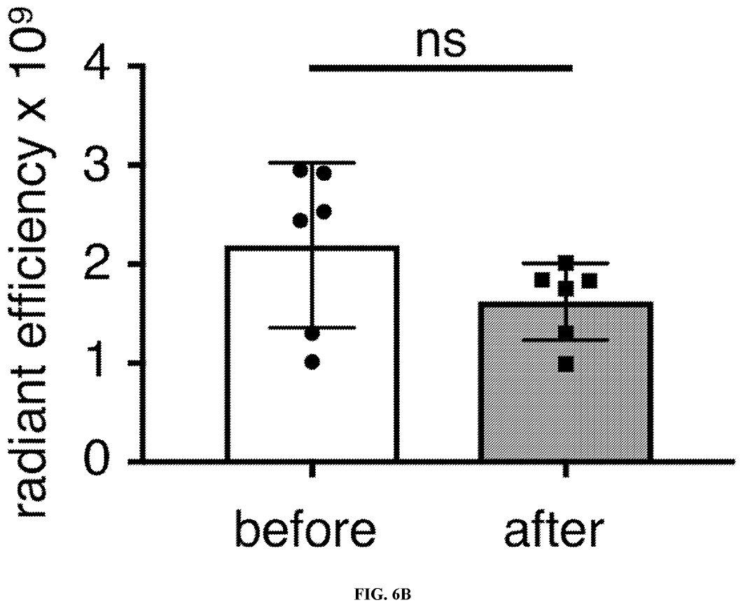

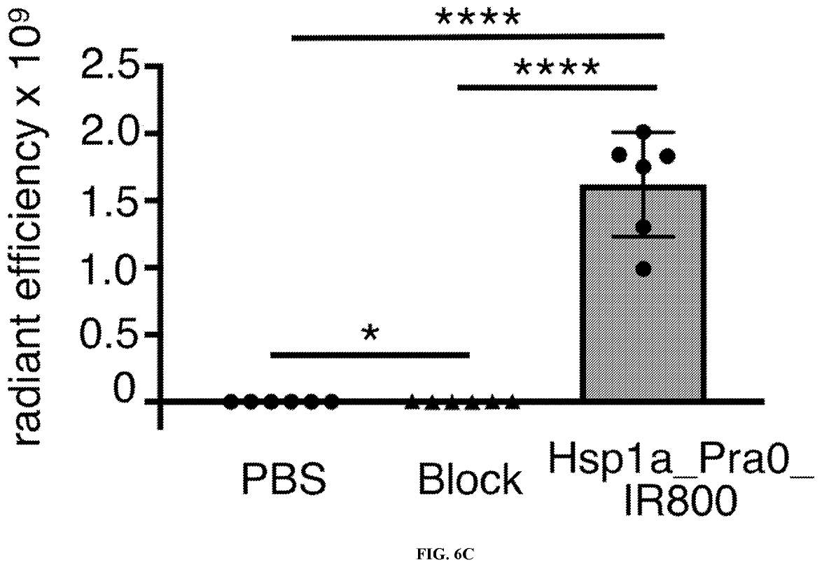

FIGS. 6A-C provides the results of imaging of fresh, unprocessed, and processed mouse sciatic nerves injected with Hsp1a_Pra0_IR800. FIG. 6A provides epifluorescence images of animals injected with PBS, Hsp1a_Pra0_IR800 (1 nmol, 10 μM of Hsp1a_Pra0_IR800 in 100 μL PBS) or a Hsp1a_Pra0_IR800/Hsp1a_Pra0 formulation (block, Hsp1a_Pra0_IR800, 10 μM, 1 nmol and Hsp1a_Pra0, 30 μM, 3 nmol in 100 μL PBS). Images were taken 30 min after tail vein injection. FIG. 6B provides a fluorescence intensity quantification of unprocessed and processed epifluorescence images of FIG. 6A before and after processing. FIG. 6C provides a fluorescence intensity quantification of FIG. 6A (left) after processing. Parametric unpaired Student's t-test was used to calculate the significance. ****P<0.0001; *P<0.01, ns, non significant. Error bars are standard deviations.

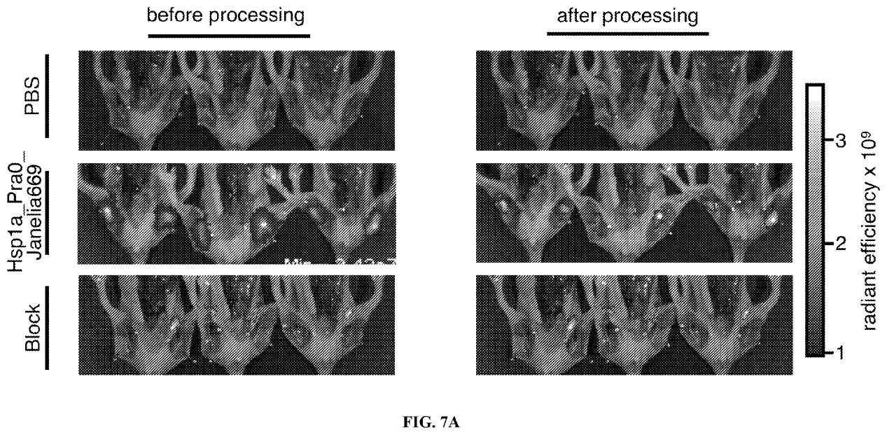

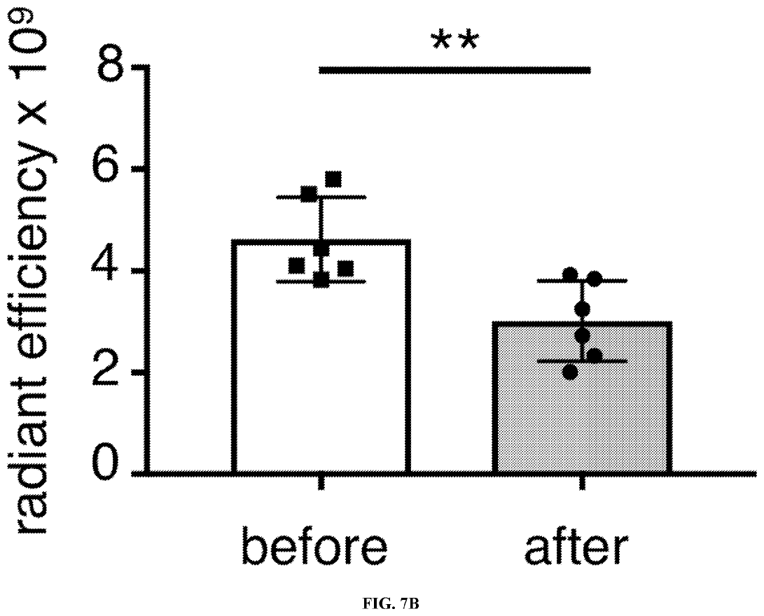

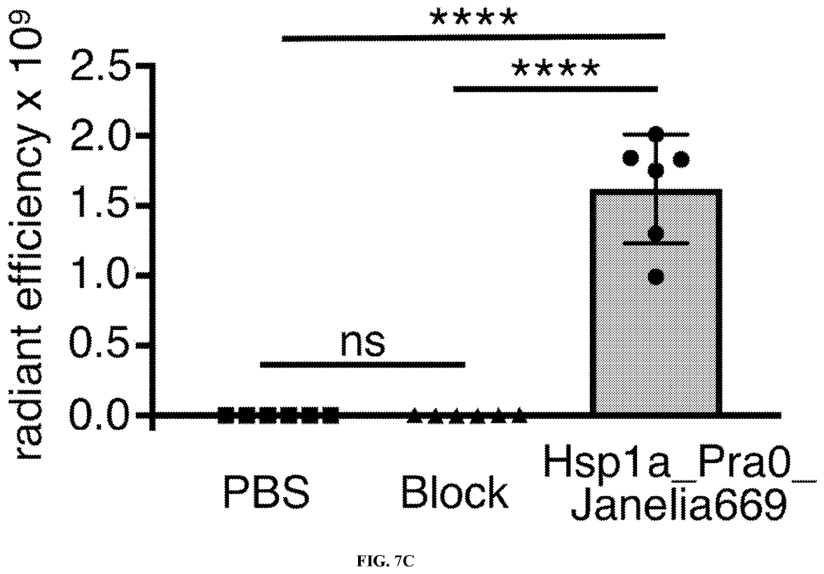

FIGS. 7A-C provides the results of imaging of fresh, unprocessed, and processed mouse sciatic nerves injected with Hsp1a_Pra0_Janelia669. FIG. 7A provides epifluorescence images of animals injected with PBS, Hsp1a_Pra0_Janelia669 (1 nmol, 10 μM of Hsp1a_Pra0_Janelia669 in 100 μL PBS) or a Hsp1a_Pra0_Janelia669/Hsp1a_Pra0 formulation (block, Hsp1a_Pra0_Janelia669, 10 μM, 1 nmol and Hsp1a_Pra0, 30 μM, 3 nmol in 100 μL PBS). Images were taken 30 min after tail vein injection. FIG. 7B provides a fluorescence intensity quantification of unprocessed and processed epifluorescence images of FIG. 7A before and after processing. FIG. 7C provides a fluorescence intensity quantification of FIG. 7A (left) after processing. Parametric unpaired Student's t-test was used to calculate the significance. ****P<0.0001; **P<0.005, ns, non significant. Error bars are standard deviations.

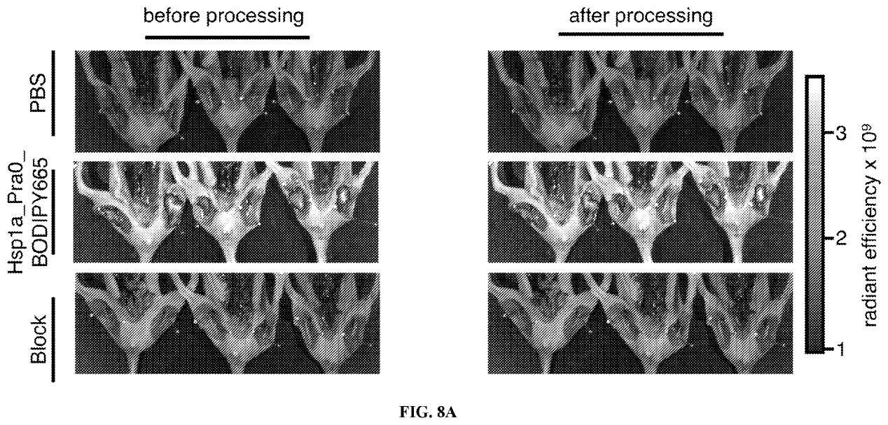

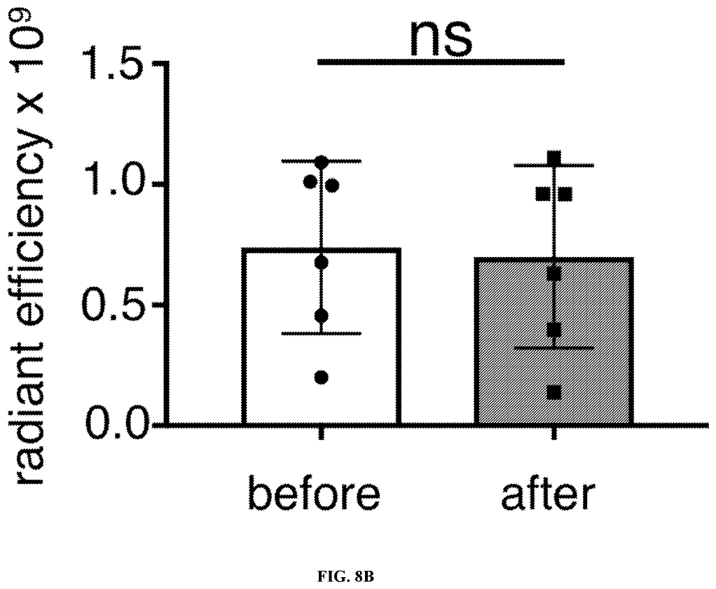

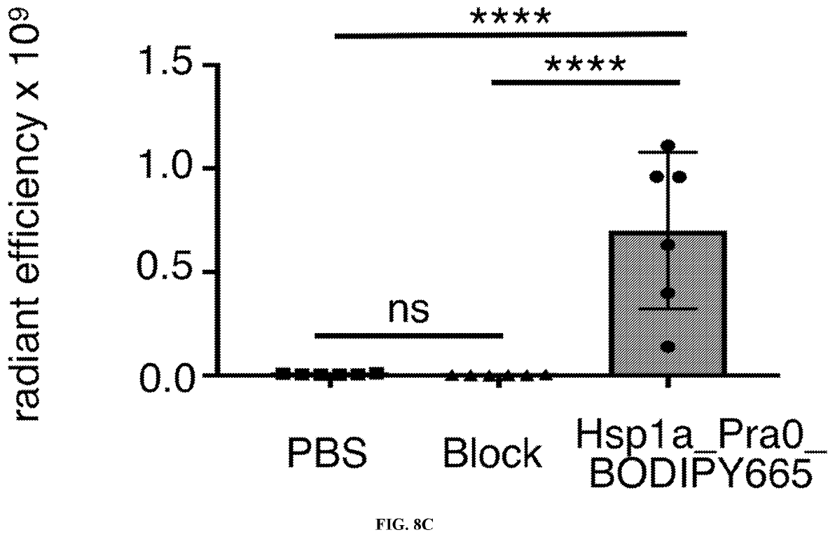

FIGS. 8A-C provides the results of imaging of fresh, unprocessed, and processed mouse sciatic nerves injected with Hsp1a_Pra0_BODIPY665. FIG. 8A provides epifluorescence images of animals injected with PBS, Hsp1a_Pra0_BODIPY665 (1 nmol, 10 μM of Hsp1a_Pra0_BODIPY665 in 100 μL PBS) or a Hsp1a_Pra0_BODIPY665/Hsp1a_Pra0 formulation (block, Hsp1a_Pra0_BODIPY665, 10 μM, 1 nmol and Hsp1a_Pra0, 30 μM, 3 nmol in 100 μL PBS). Images were taken 30 min after tail vein injection. FIG. 8B provides a fluorescence intensity quantification of unprocessed and processed epifluorescence images of FIG. 8A before and after processing. FIG. 8C provides a fluorescence intensity quantification of FIG. 8A (left) after processing. Parametric unpaired Student's t-test was used to calculate the significance. ****P<0.0001, ns, non significant. Error bars are standard deviations.

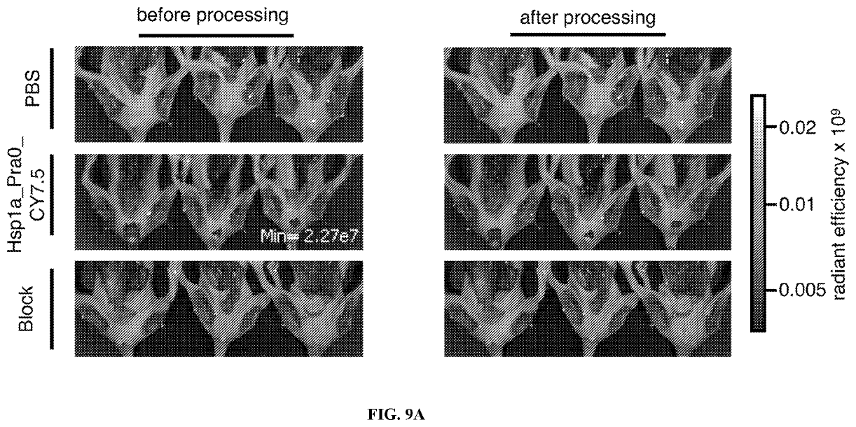

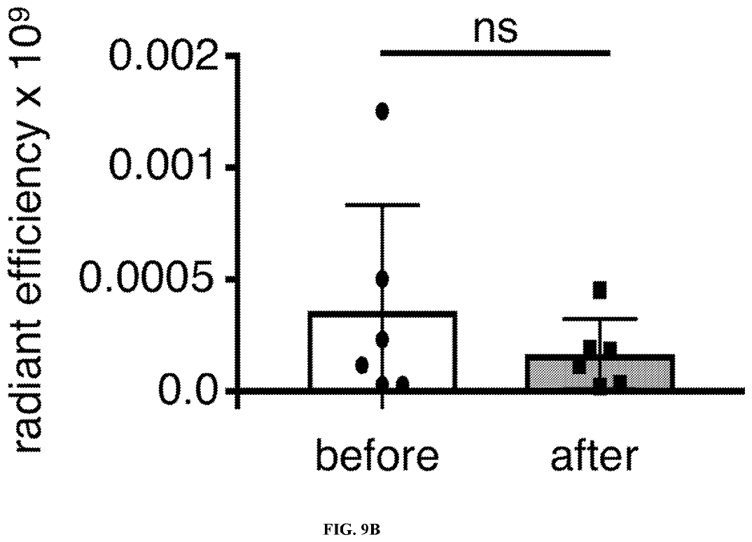

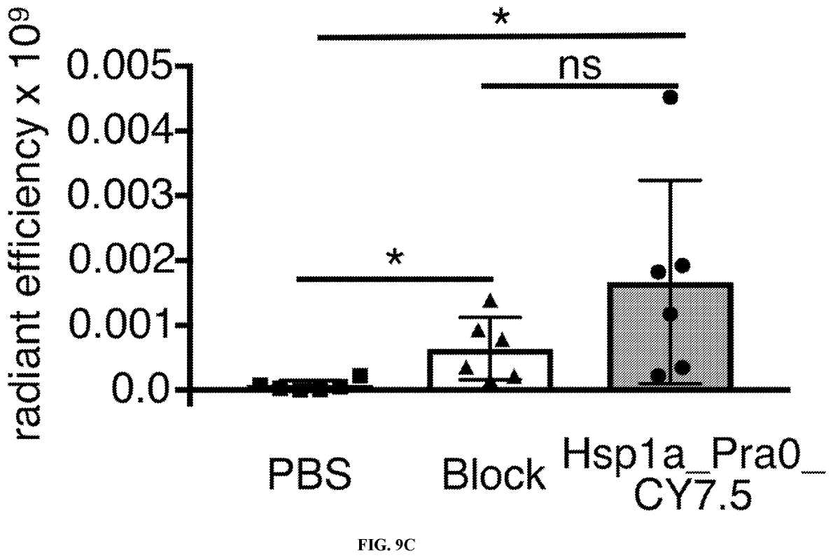

FIGS. 9A-C provides the results of imaging of fresh, unprocessed, and processed mouse sciatic nerves injected with Hsp1a_Pra0_CY7.5. FIG. 9A provides epifluorescence images of animals injected with PBS, Hsp1a_Pra0_CY7.5 (1 nmol, 10 μM of Hsp1a_Pra0_CY7.5 in 100 μL PBS) or a Hsp1a_Pra0_CY7.5/Hsp1a_Pra0 formulation (block, Hsp1a_Pra0_CY7.5, 10 μM, 1 nmol and Hsp1a_Pra0, 30 μM, 3 nmol in 100 μL PBS). Images were taken 30 min after tail vein injection. FIG. 9B provides a fluorescence intensity quantification of unprocessed and processed epifluorescence images of FIG. 9A before and after processing. FIG. 9C provides a fluorescence intensity quantification of FIG. 9A (left) after processing. Parametric unpaired Student's t-test was used to calculate the significance. *P<0.01, ns, non significant. Error bars are standard deviations.

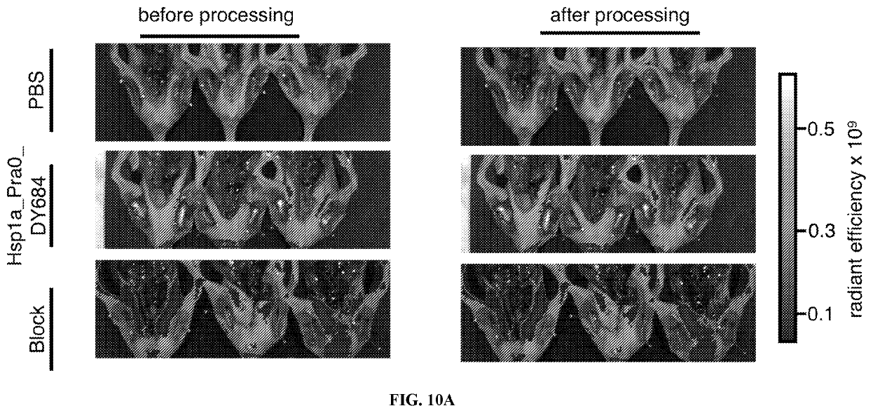

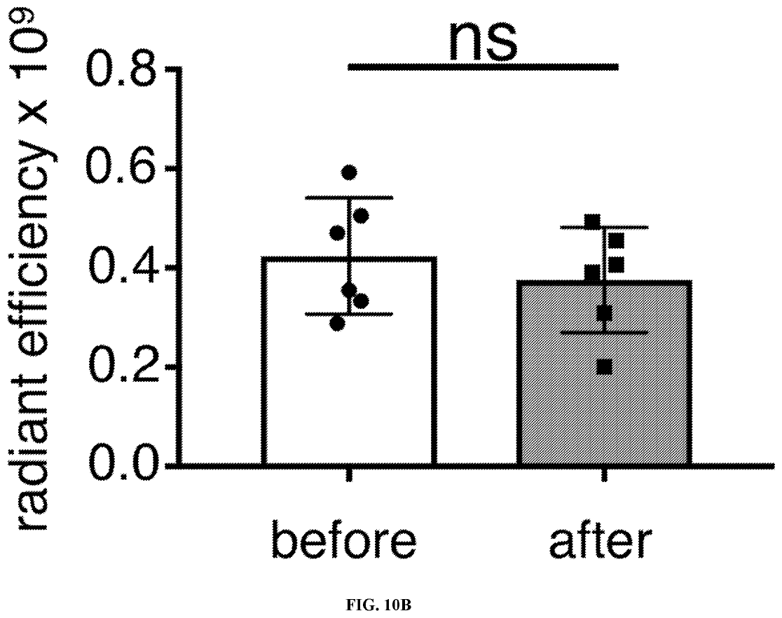

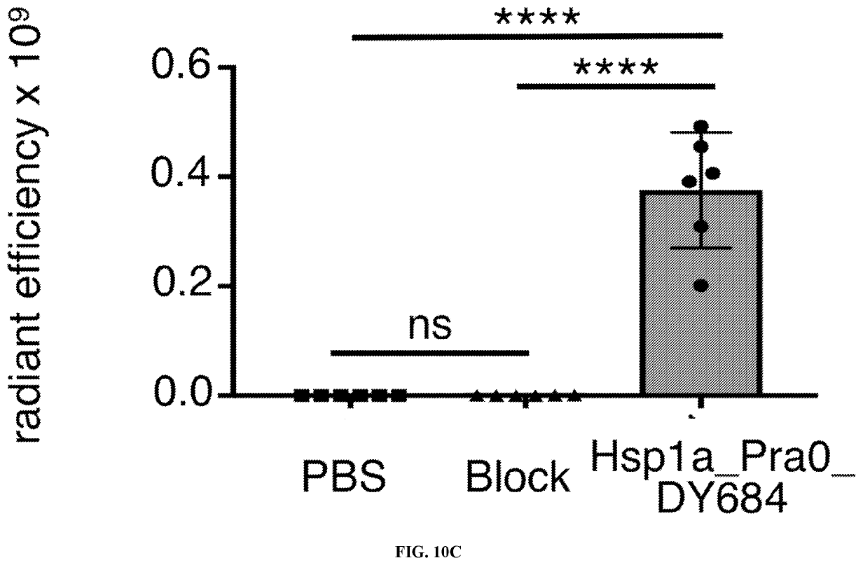

FIGS. 10A-C provides the results of imaging of fresh, unprocessed, and processed mouse sciatic nerves injected with Hsp1a_Pra0_DY684. FIG. 10A provides epifluorescence images of animals injected with PBS, Hsp1a_Pra0_DY684 (1 nmol, 10 μM of Hsp1a_Pra0_DY684 in 100 μL PBS) or a Hsp1a_Pra0_DY684/Hsp1a_Pra0 formulation (block, Hsp1a_Pra0_DY684, 10 μM, 1 nmol and Hsp1a_Pra0, 30 μM, 3 nmol in 100 μL PBS). Images were taken 30 min after tail vein injection. FIG. 10B provides a fluorescence intensity quantification of unprocessed and processed epifluorescence images of FIG. 10A before and after processing. FIG. 10C provides a fluorescence intensity quantification of FIG. 10A (left) after processing. Parametric unpaired Student's t-test was used to calculate the significance. ****P<0.0001, ns, non significant. Error bars are standard deviations.

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such
A. A compound of Formula I

(I)
(SEQ ID NO: 1)
χ¹-YCQ-χ²-FLWTCDSERPCCEGLVCRLWC-χ³-IN-NH₂,

or a conservative amino acid substitution variant thereof, a pharmaceutically acceptable salt thereof, and/or a solvate thereof,
wherein
   [symbol] ¹ is H of the alpha amine of Y, a chelator covalently conjugated to the alpha amine of Y and optionally wherein the chelator is chelating a radionuclide cation (“chelated radionuclide cation”), R¹ covalently conjugated to the alpha amine of Y, or

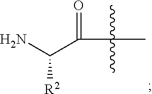

[symbol] ² is

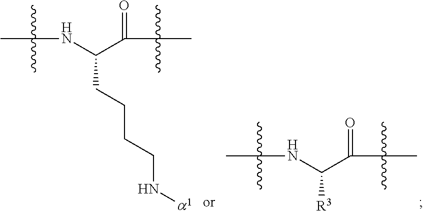

[symbol] ³ is

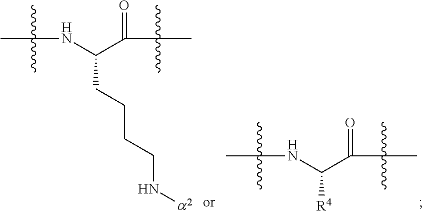

α¹ and α² are each independently H or a chelator optionally chelating a radionuclide cation (“chelated radionuclide cation”);
R¹, R², R³, and R⁴ are each independently

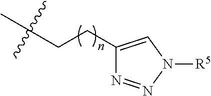

where n is 0, 1, 2, 3, or 4, R⁵ is independently at each occurrence a fluorophore, —(CH₂)ₘ-¹⁸F, an ¹⁸F-substituted aryl, or an ¹⁸F-substituted aralkyl, and m is independently at each occurrence 2, 3, 4, 5, 6, 7, or 8;
provided that [symbol] ¹ is not H of the alpha amine of Y when both α¹ and α² are each independently H.
B. The compound of Paragraph A, wherein the fluorophore independently at each occurrence arises from IR780, IR800, DY-684, DY-700, Janelia669, BODIPY, BODIPY665, sulfo-CY5, CY5.5, CY7, CY7.5, ICG, IR140, or DiR.
C. The compound of Paragraph A or Paragraph B, wherein the fluorophore is independently at each occurrence selected from

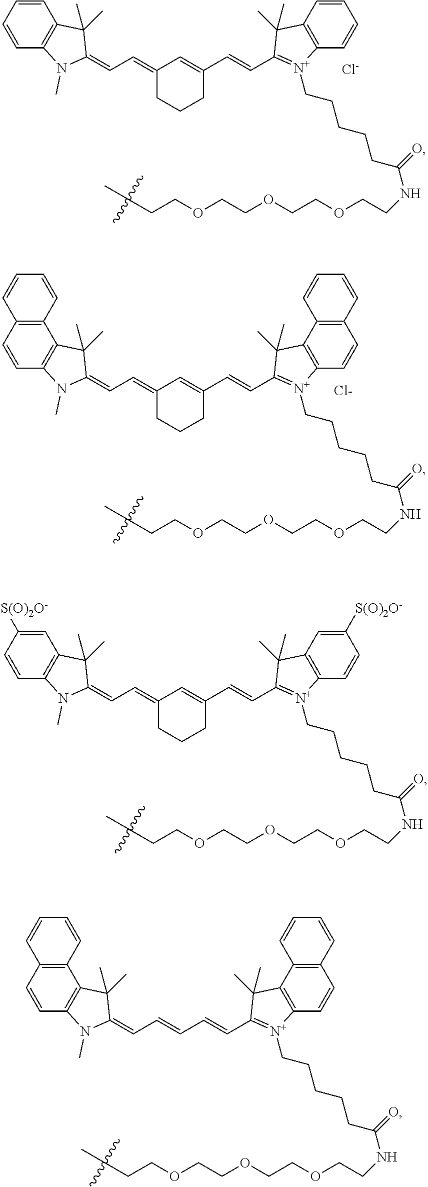

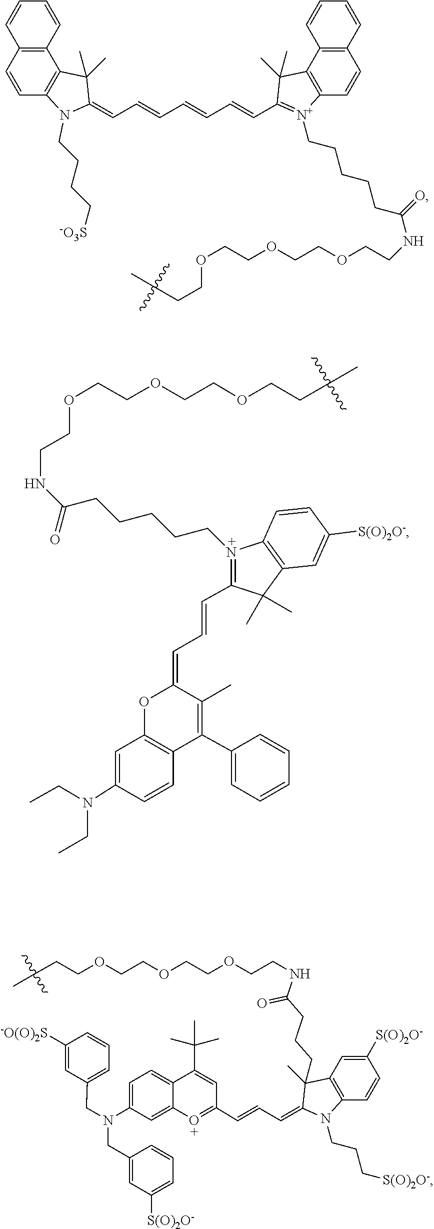

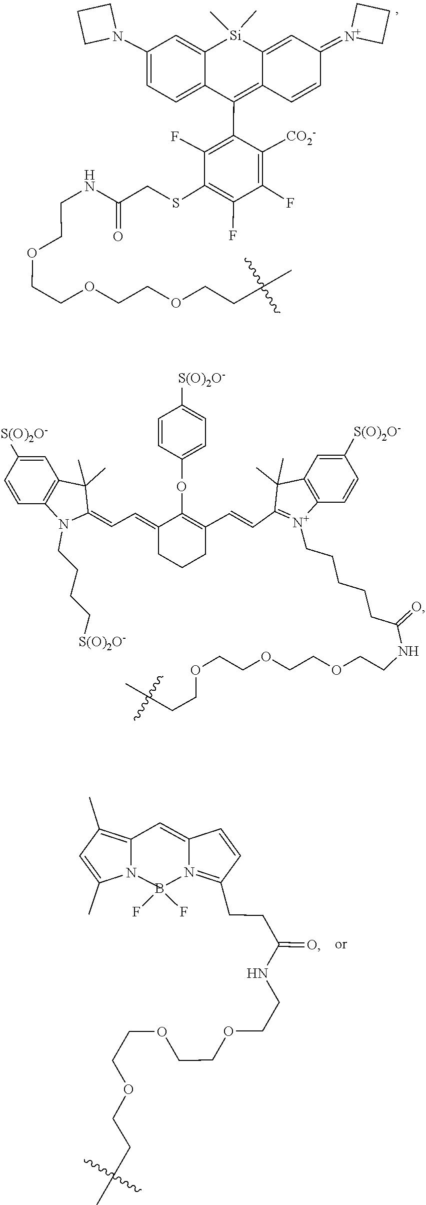

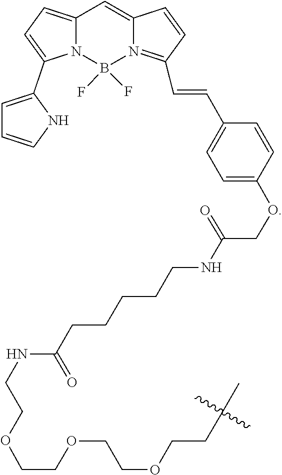

D. The compound of any one of Paragraphs A-C, wherein the chelator is independently at each occurrence

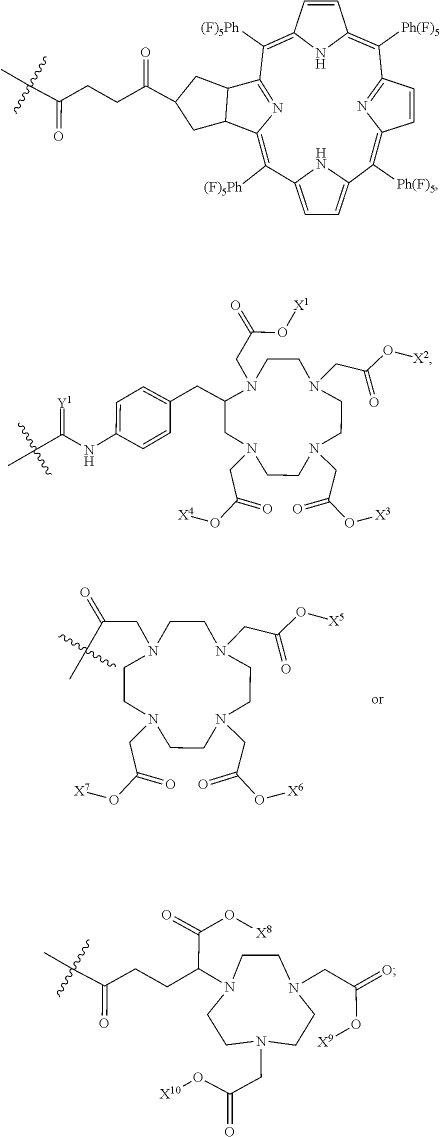

where
   X¹, X², X³, X⁴, X⁵, X⁶, X⁷, X⁸, X⁹, and X¹⁰ are each independently a lone pair of electrons (i.e., providing an oxygen anion) or H; and
   Y¹ is S or O.
E. The compound of any one of Paragraphs A-D, wherein the chelator is chelating a radionuclide cation (“chelated radionuclide cation”), and wherein the chelator and chelated radionuclide cation together is independently at each occurrence

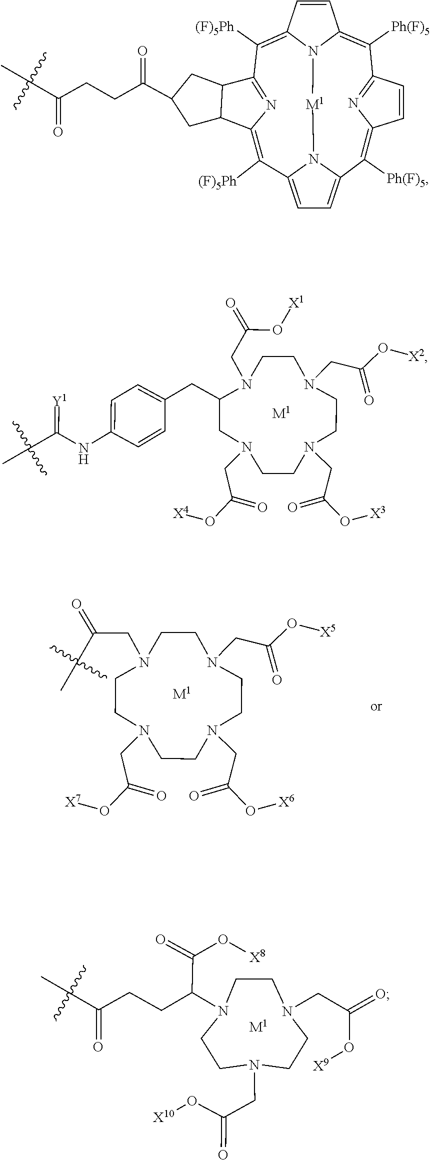

where
   M¹ is independently at each occurrence the chelated radionuclide cation;
   X¹, X², X³, X⁴, X⁵, X⁶, X⁷, X⁸, X⁹, and X¹⁰ are each independently a lone pair of electrons (i.e., providing an oxygen anion) or H; and
   Y¹ is S or O.
F. The compound of any one of Paragraphs A-E, wherein the chelated radionuclide cation is independently at each occurrence ⁸⁹Zr, ⁶⁷Ga, ⁶⁸Ga, ⁸⁶Y ⁹⁰Y, ⁸⁹Sr, ¹⁶⁵Dy, ¹¹¹In, ¹⁶Re, ¹⁸⁸Re, ¹⁷⁷Lu, ⁶⁷Cu, ⁶²Cu, or ⁶⁴Cu.
G. The compound of any one of Paragraphs A-F, wherein at least one of R¹, R², R³, and R⁴ is

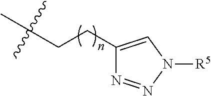

and at least one R⁵ is —(CH₂)ₘ-¹⁸F, an ¹⁸F-substituted aryl, or an ¹⁸F-substituted aralkyl.
H. A composition comprising the compound of any one of Paragraphs A-G and a pharmaceutically acceptable carrier.
I. A pharmaceutical composition comprising
   an effective amount of the compound of any one of Paragraphs A-G for imaging peripheral neurons in a subject; and
   a pharmaceutically acceptable carrier.
J. A method comprising
   administering a compound of any one of Paragraphs A-G to a subject; and
   subsequent to the administering, detecting one or more of fluorescence emission, positron emission, gamma rays from positron emission and annihilation, and Cerenkov radiation due to positron emission.
K. The method of Paragraph J, wherein the method comprises administering an imaging-effective amount of the compound to the subject for imaging peripheral neurons.
L. The method of Paragraph J or Paragraph K, wherein the detecting comprises widefield intraoperative imaging, mesoscopic intraoperative imaging, microscopic intraoperative imaging, laparoscopic intraoperative imaging, or a combination of any two or more thereof.
M. The method of any one of Paragraphs J-L, wherein administering the compound comprises parenteral administration.
N. A method of obtaining an image, the method comprising
   administering an imaging-effective amount of a compound of any one of Paragraphs A-G for imaging peripheral neurons to a subject; and
   subsequent to the administering, detecting one or more of fluorescence emission, positron emission, gamma rays from positron emission and annihilation, and Cerenkov radiation due to positron emission.
O. The method of Paragraph N, wherein the detecting comprises widefield intraoperative imaging, mesoscopic intraoperative imaging, microscopic intraoperative imaging, laparoscopic intraoperative imaging, or a combination of any two or more thereof.
P. The method of Paragraph N or Paragraph O, wherein administering the compound comprises parenteral administration.

Other embodiments are set forth in the following claims.

1. A compound of Formula I
(I)
(SEQ ID NO: 1)
χ¹-YCQ-χ²-FLWTCDSERPCCEGLVCRLWC-χ³-IN-NH₂,
or a conservative amino acid substitution variant thereof, a pharmaceutically acceptable salt thereof, and/or a solvate thereof,
wherein [symbol]
¹ is H of the alpha amine of Y, a chelator covalently conjugated to the alpha amine of Y and optionally wherein the chelator is chelating a radionuclide cation (“chelated radionuclide cation”), R¹ covalently conjugated to the alpha amine of Y, or 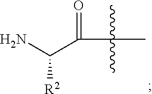 [symbol]
² is 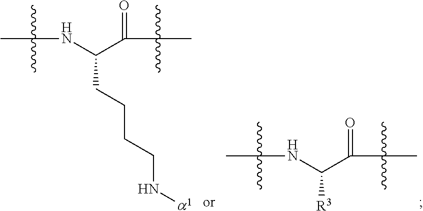 [symbol]
³ is 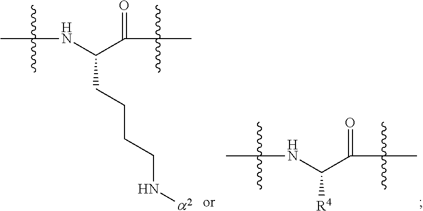
α¹ and α² are each independently H or a chelator optionally chelating a radionuclide cation (“chelated radionuclide cation”);
R¹, R², R³, and R⁴ are each independently 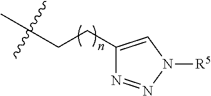
where n is 0, 1, 2, 3, or 4, R⁵ is independently at each occurrence a fluorophore, —(CH₂)ₘ-¹⁸F, an ¹⁸F-substituted aryl, or an ¹⁸F-substituted aralkyl, and m is independently at [symbol] each occurrence 2, 3, 4, 5, 6, 7, or 8;
provided that [symbol] ¹ is not H of the alpha amine of Y when both α¹ and α² are each independently H.

2. The compound of claim 1, wherein the fluorophore independently at each occurrence arises from IR780, IR800, DY-684, DY-700, Janelia669, BODIPY, BODIPY665, sulfo-CY5, CY5.5, CY7, CY7.5, ICG, IR140, or DiR.

3. The compound of claim 1, wherein the fluorophore is independently at each occurrence selected from

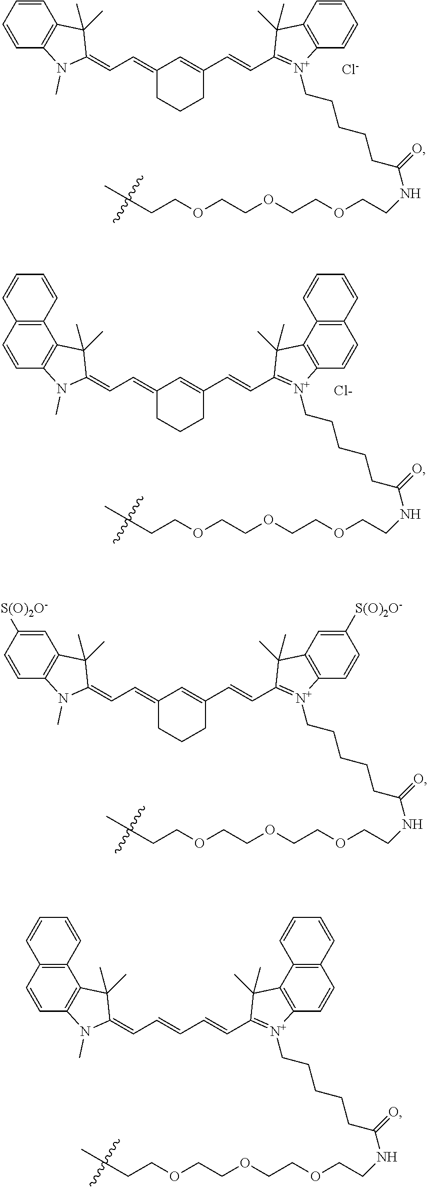

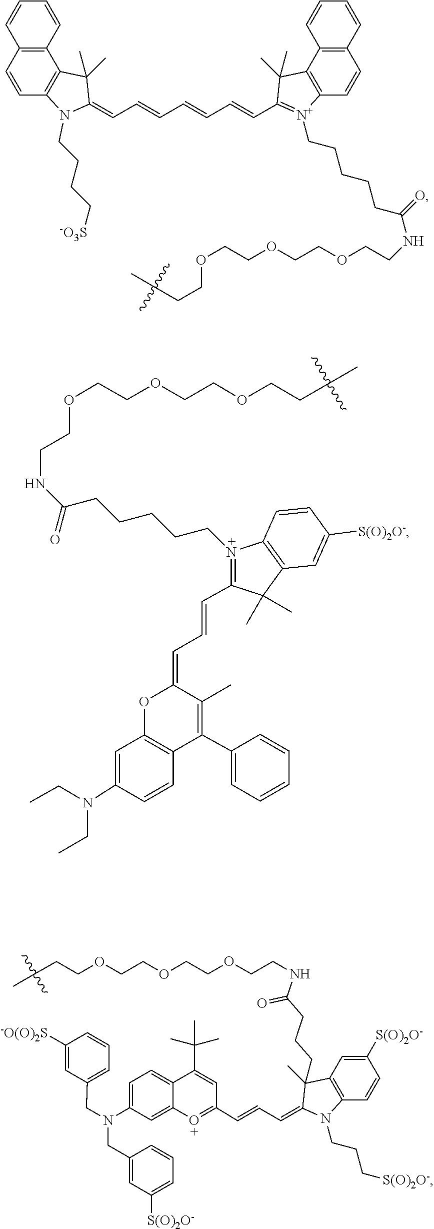

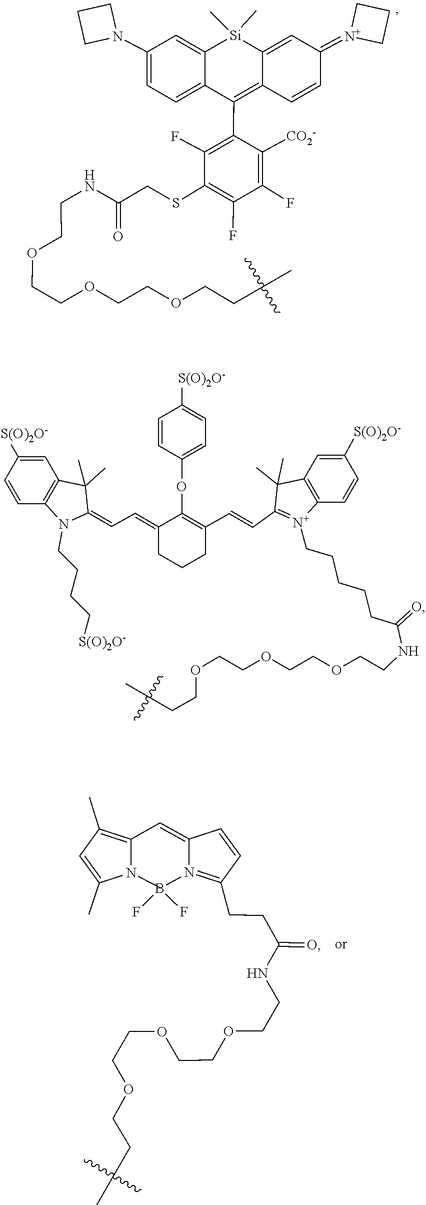

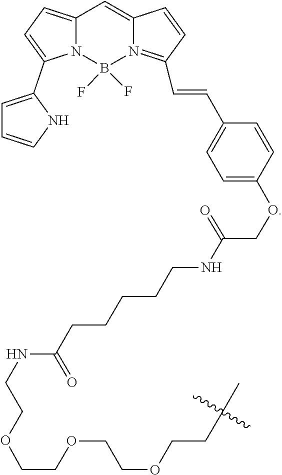

4. The compound of claim 1, wherein the chelator is independently at each occurrence 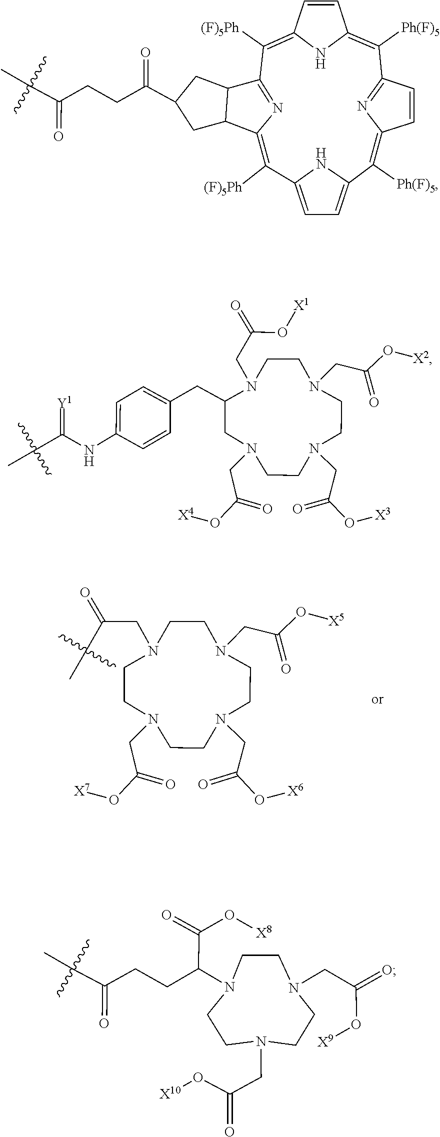
where
X¹, X², X³, X⁴, X⁵, X⁶, X⁷, X⁸, X⁹, and X¹⁰ are each independently a lone pair of electrons or H; and
Y¹ is S or O.

5. The compound of claim 4, wherein the chelator is chelating a radionuclide cation (“chelated radionuclide cation”), and wherein the chelator and chelated radionuclide cation together is independently at each occurrence 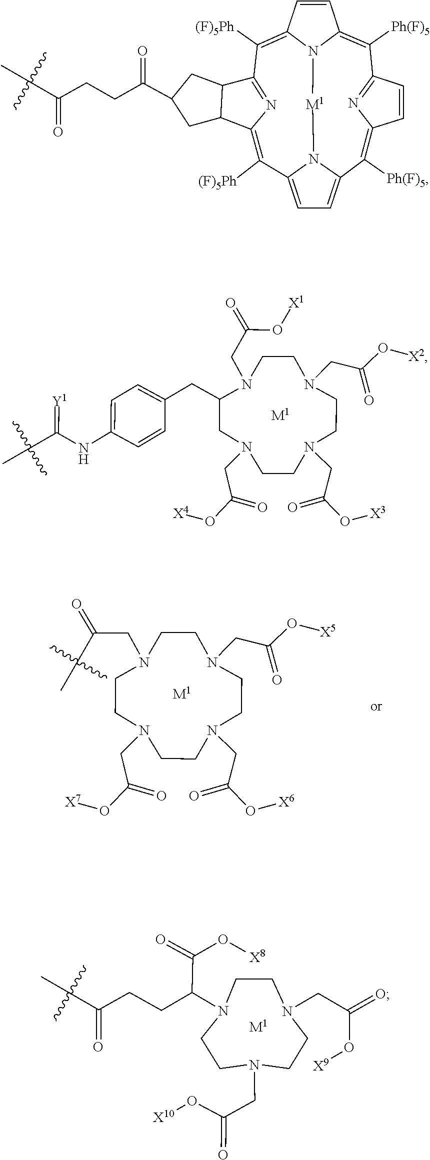
where M¹ is independently at each occurrence a chelated radionuclide cation; X¹, X², X³, X⁴, X⁵, X⁶, X⁷, X⁸, X⁹, and X¹⁰ are each independently a lone pair of electrons or H; and Y¹ is S or O.

6. The compound of claim 5, wherein the chelated radionuclide cation is independently at each occurrence ⁸⁹Zr, ⁶⁷Ga, ⁶⁸Ga, ⁸⁶Y ⁹⁰Y, ⁸⁹Sr, ¹⁶⁵Dy, ¹¹¹In, ¹⁶Re, ¹⁸⁸Re, ¹⁷⁷Lu, ⁶⁷Cu, ⁶²Cu, or ⁶⁴Cu.

7. The compound of claim 1, wherein at least one of R¹, R², R³, and R⁴ is 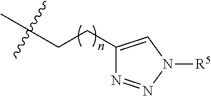
and at least one R⁵ is —(CH₂)ₘ-¹⁸F, an ¹⁸F-substituted aryl, or an ¹⁸F-substituted aralkyl.

8. A composition comprising
the compound of claim 1; and
a pharmaceutically acceptable carrier.

9. A pharmaceutical composition comprising
an effective amount of the compound of claim 1 for imaging peripheral neurons in a subject; and
a pharmaceutically acceptable carrier.

10. A method comprising
administering a compound of claim 1 to a subject; and
subsequent to the administering, detecting one or more of fluorescence emission, positron emission, gamma rays from positron emission and annihilation, and Cerenkov radiation due to positron emission.

11. The method of claim 10, wherein the method comprises administering an imaging-effective amount of the compound to the subject for imaging peripheral neurons.

12. The method of claim 10, wherein the detecting comprises widefield intraoperative imaging, mesoscopic intraoperative imaging, microscopic intraoperative imaging, laparoscopic intraoperative imaging, or a combination of any two or more thereof.

13. The method of claim 10, wherein administering the compound comprises parenteral administration.

14. The method of claim 11, wherein administering the compound comprises parenteral administration.

15. The method of claim 12, wherein administering the compound comprises parenteral administration.

16. A method of obtaining an image, the method comprising
administering an imaging-effective amount of a compound of claim 1 for imaging peripheral neurons to a subject; and
subsequent to the administering, detecting one or more of fluorescence emission, positron emission, gamma rays from positron emission and annihilation, and Cerenkov radiation due to positron emission.

17. The method of claim 16, wherein the detecting comprises widefield intraoperative imaging, mesoscopic intraoperative imaging, microscopic intraoperative imaging, laparoscopic intraoperative imaging, or a combination of any two or more thereof.

18. The method of claim 16, wherein administering the compound comprises parenteral administration.

19. The method of claim 17, wherein administering the compound comprises parenteral administration.


SEQUENCE LISTING (5 sequences)

SEQ ID NO: 1 - protein, 29 aa, Artificial Sequence
  Description of Artificial Sequence Synthetic peptide
  MOD_RES (1)..(1): Any amino acid or absent
  MOD_RES (5)..(5): Lysine or absent
  MOD_RES (27)..(27): Lysine or absent
  See specification as filed for detailed description of substitutions and preferred embodiments
XYCQXFLWTCDSERPCCEGLVCRLWCXIN

SEQ ID NO: 2 - protein, 28 aa, Artificial Sequence
  Description of Artificial Sequence Synthetic peptide
YCQKFLWTCDSERPCCEGLVCRLWCKIN

SEQ ID NO: 3 - protein, 29 aa, Artificial Sequence
  Description of Artificial Sequence Synthetic peptide
  MOD_RES (1)..(1): L-propargylglycine
XYCQKFLWTCDSERPCCEGLVCRLWCKIN

SEQ ID NO: 4 - protein, 28 aa, Artificial Sequence
  Description of Artificial Sequence Synthetic peptide
  MOD_RES (4)..(4): L-propargylglycine
YCQXFLWTCDSERPCCEGLVCRLWCKIN

SEQ ID NO: 5 - protein, 28 aa, Artificial Sequence
  Description of Artificial Sequence Synthetic peptide
  MOD_RES (26)..(26): L-propargylglycine
YCQKFLWTCDSERPCCEGLVCRLWCXIN